(19) United States
(12) Patent Application Publication
(10) Pub. No.: US 2022/0408703 A1
(43) Pub. Date: Dec. 29, 2022

(54) IMAGING INDIVIDUAL HIPPOCAMPAL SEIZURES AND THE LONG-TERM IMPACT OF REPEATED SEIZURES

(71) Applicant: The Board of Trustees of the Leland Stanford Junior University, Stanford, CA (US)

(72) Inventors: Jin Hyung Lee, Stanford, CA (US); Mankin Choy, Redwood City, CA (US); Ben A. Duffy, Palo Alto, CA (US)

(21) Appl. No.: 17/780,170

(22) PCT Filed: Dec. 7, 2020
(86) PCT No.: PCT/US2020/063579

§ 371 Date: May 26, 2022

Related U.S. Application Data
(60) Provisional application No. 62/945,012, filed on Dec. 6, 2019.

(51) Int. Cl. A01K 67/027; A61K 49/00

(52) U.S. Cl. CPC A01K 67/0275; A61K 49/0008; A01K 2217/052; A01K 2267/0356

(57) ABSTRACT

It is shown that ventral hippocampal kindling results in functional reorganization of the ventral hippocampal excitatory circuits. Most pronounced is the connectivity to the medial prefrontal cortex, with increased volume of activation on fMRI and increased amplitude of activation on electrophysiology. There is evidence of increased anxiety following kindling Methods are provided for simultaneous LFP-fMRI to image single seizures Imaging the spatiotemporal dynamics of individual seizures enables characterization of propagation patterns of focal and secondary-generalized seizures, that provide for targeted intervention.

### CROSS REFERENCE TO RELATED APPLICATION

This application claims priority to U.S. Provisional Application No. 62/945,012, filed Dec. 6, 2019, which is incorporated herein in its entirety for all purpose.

### BACKGROUND

When seizure activity invades critical circuits involved in consciousness or cardiorespiratory regulation, the risk of accidents or death increases, respectively. How such seizures evolve and propagate remain poorly understood, but there is evidence of both cortical and subcortical circuit involvement. Standard methods of recording seizures, such as electrophysiology and optical imaging, have limited spatial coverage and cannot capture the evolution of the brain-wide activity associated with certain events. For example, focal to bilateral tonic-clonic (FBTC) seizures, formerly known as secondarily generalized seizures, are difficult to treat and significantly impact patients' quality of life and safety. fMRI offers brain-wide information and has been used to visualize both focal and generalized-onset seizures in humans and in animals, but its use for visualizing FBTC seizures has been difficult due to associated motor activity that result in motion artefacts.

The relationship behind seizures, kindling, and sub-threshold activity has long been difficult to understand. This is critical for epilepsy diagnosis and treatment. The methodology provided herein can be used to diagnose and make treatment decisions for epilepsy. This is a new method that can define concrete relationships among seizures, kindling, and sub-threshold activity for the first time.

### SUMMARY

Methods and models are provided for the analysis of events associated with seizures in the brain, e.g. events that model epileptic seizures; including assessing the impacts of functional and electrical neural circuit changes. The methods and models may include, for example, one or more of analysis of single seizures, triggering by sub-threshold stimulus, analysis of focal to bilateral tonic-clonic (FBTC) seizures, analysis of excitatory ventral hippocampal (VH) networks, analysis of migrating seizure cores, and the like.

In some embodiments, methods and models are provided for identification and localization of migrating seizure cores in an individual. In some embodiments, methods and models are provided for the design of therapeutic agents to treat epileptic seizures, including without limitation FBTC seizures. In some embodiments the identification of migrating seizure cores is used in the identification of seizure onset zones. Methods of analysis may include, without limitation, determining electrophysiology, e.g. local field potentials (LFP), and functional magnetic resonance imaging (fMRI). Models may include, without limitation, optogenetic models, and the use of optogenetic stimulation for kindling and seizure induction. Other methods of stimulation also find use, e.g. electric, magnetic, pharmacologic, etc. stimulation. Stimulation methods are preferred that can be applied to generate a single seizure, e.g. in the hippocampal region. Stimulation methods include sub-threshold activity.

The data provided herein demonstrates the use of a kindling and seizure induction model that can be analyzed with simultaneous electrophysiology and functional MRI. To image single seizures with simultaneous LFP-fMRI in an animal model, the animal may be sedated and treated with a short-acting neuromuscular blocker to abolish motion during imaging of seizures, exemplary agents for this purpose include, without limitation, dexmedetomidine sedation and vecuronium. Through imaging of individual seizures events associated with seizures can be analyzed in fine detail. For example, a core of slow migrating activity in the hippocampus is shown to provide a novel seizure propagation and generalization mechanism. Models include a kindled animal brain, e.g. a live animal, which may be a mammal, e.g. a rodent such as a rat, mouse, etc., non-human primate, and the like.

In some embodiments an optogenetic kindling model for seizures is provided, wherein electrographic seizures are induced in an animal model by cell-type specific, optogenetic stimulations, which animal then provides for reliable induction of FBTC seizures over an extended period of time, where an extended period of time may be up to two weeks, up to 3 weeks, up to 4 weeks, up to 2 months, up to 3 months, or more. The light-activated polypeptide for stimulation may be, for example, a channelrhodopsin, including without limitation CHR2. The light activated polypeptide may be operably linked to a promoter expressed in excitatory hippocampal neurons. Stimulation paradigms include a series of short and mild stimulations below the threshold to trigger seizures, e.g. of around about 10 Hz, to evaluate underlying functional circuit changes. A long and intense stimulation of about 40 Hz can be used to evaluate seizure circuit dynamics. Simultaneous electrophysiology and fMRI can be used to determine the effect of kindling excitatory neurons in the ventral hippocampus and seizure induction. Imaging brain-wide network dynamics of single induced seizures shows focal and FBTC seizure propagation.

The models provided herein are useful in the design and testing of therapeutic interventions, e.g. surgery, pharmacologic intervention, and the like, where the effect of a therapeutic intervention on seizure induction and propagation can be determined. The models are also useful in the design of drugs for epilepsy co-morbidities, e.g. the largest activity changes are in the medial prefrontal cortex (mePFC), indicating an increased excitatory relationship between ventral hippocampus (vHip) and mePFC. Therapies designed to target vHip-mePFC circuit dysfunction can reduce comorbidities associated with epilepsy, including anxiety and cognitive defects. In some embodiments these variables of seizure propagation are used to guide surgical targeting and therapy development for epilepsy. Included in the findings herein is the showing of a slow migrating core of high amplitude activity that can accompany seizures, and is frequently observed prior to seizures.

In some embodiments, improved methods of epilepsy surgery are provided. Such methods, as known in the art, require a seizure onset zone (SOZ) to be reliably localized. It is shown herein that a migrating hippocampal core provides a fundamental mechanism for seizure propagation, which can affect SOZ localization. The SOZ may not be active throughout the seizure; regional patterns of seizure activity can change quickly; and regions with the highest activity are often not the SOZ. Standard clinical SOZ identification methods include, for example, single-photon emission computed tomography (SPECT), electrophysiology and the like. Improved methods for detecting SOZ reflect detection of the migrating core to improve SOZ localization.

### BRIEF DESCRIPTION OF DRAWINGS

FIG. 1: Optogenetic ventral hippocampal kindling persists for many months and without changes in hippocampal volume. (A) CamKII cells in the ventral hippocampus were targeted for optogenetic kindling and an optrode was implanted for stimulation and electrophysiology. An electrode was implanted into the ipsilateral medial prefrontal cortex for electrophysiology. Right Panel: Confocal images with localization of ChR2-eYFP expression to the ventral hippocampus. (B) For optogenetic kindling, rats were stimulated every other day with up to 12 stimulations a day or until a Racine stage 5 seizure was observed. Stimulations continued until criterion for kindling was reached: a stage 5 seizure within the first three stimulations of the day. (C) 83% (10/12) of rats were kindled by day 7, and all were kindled by day 11. (D) Behavior scores for every stimulation in each rat undergoing kindling shows successful kindling. (E) Total number of stimulations each rat received to acquire kindling. (F) There is no significant change in hippocampal volumes pre and post in non-kindled and kindled animals. Left panel: ipsilateral hippocampal volume changes, t-test (t=−0.385, p=0.703), Right panel: contralateral hippocampal volume changes, t-test (t=−1.361, p=0.187). (G) A subgroup of rats was tested 3 weeks and then 12 weeks following kindling acquisition to determine if the animals retained the kindling phenotype. Rats were stimulated 3 times. All kindled animals had Racine stage 5 seizures whereas no control rats displayed any seizure-associated Racine behaviors (n=5 per group) showing persistent change resulting from kindling. All data presented as mean±s.e.m. A threshold of p<0.05 was used for determination of statistical significance.

FIG. 2: Hippocampal-kindling results in brain-wide changes in hippocampal connectivity and heightened anxiety. (A) Ventral hippocampal CamKII cells were targeted for stimulation with electrodes in ipsilateral ventral hippocampus (iVHip) and ipsilateral medial prefrontal cortex (iMePFC) for LFP recordings. fMRI imaged 27 slices across the brain. (B) Simultaneous LFP-fMRI was used to assess 10 Hz VHip connectivity before and after kindling and in non-kindled control animals (n=12 in each group). For each scan, a 5 s stimulation delivered at 10 Hz were given once every minute for six cycles. (C) Statistical t-maps for cage-mate, age-matched controls (left panel) and in kindled (right panel) animals with a t-threshold corresponding to p<0.001. Note that activity in the controls was mostly restricted to iVHip, iDHip, iMePFC, iAmyg, and iSept, whereas activity in the kindled rats include these areas and expands to others too. (D) Brain-wide regional activation volumes in non-kindled and kindled rats. Regional activation volumes were quantified in individual animals and expressed as proportion of a region activated. Volumes were sorted from largest to smallest mean volumes of kindled rats. Seven regions were significantly different between controls and kindled animals (t-test with adjusted p-values using Bonferroni-Holm's correction for multiple comparisons). The largest effect was observed in the iMePFC, followed by cMePFC, iFrAssC, iTeAssC, iOrFrC, ilnsC, and finally iStria. (E) Regional CBV-fMRI peak amplitude response to 10 Hz stimulation in non-kindled and kindled animals. The five largest active regions (% of ROI active) in non-kindled animals were selected to determine the impact of kindling on the non-kindled network and CBV amplitudes were inverted for ease of interpretation. Following kindling, significant increases in amplitudes were observed in iMePFC and iAmyg. Each grey circle represents data from a single animal. (F) Simultaneously acquired LFP response in iVHip and iMePFC from a single rat before (left panel) and after kindling (right panel). Blue bar indicates 10 Hz optogenetic stimulation. Note increase in iMePFC LFP response following kindling. LFPs were gradient-artefact corrected and bandpass-filtered at 8-12 Hz. (G) Group analysis of LFP response in non-kindled and kindled rats. Left panel: iVHip LFP response to 10 Hz stimulation did not differ between pre and post conditions in the control and kindled animals (n=12 per group, F=0.59, p=0.81, two-way repeated measures ANOVA). Right panel: iMePFC LFP response to 10 Hz stimulation was increased following kindling when compared to control animals (n=11 per group, F=5.39, p=0.031, two-way repeated measures ANOVA). Post-hoc analysis indicated that there was a 6.4±2.6 fold increase following kindling (paired t-test, t=2.47, p=0.033). To estimate LFP power, the ratio between LFP power during stimulation and 5 s immediately before stimulation was calculated. For each timepoint per animal, the median of the ratio from 6-18 stimulation blocks was used to assess time-dependent group effects. For each block, normalized power was calculated from the stimulation block relative to the 5 s prior to stimulation onset. Two animals were removed for the prefrontal cortical analysis due to a broken electrode (1 control), and contamination with artefact (1 kindled). Please see the supplementary section for data from these animals. (H) 10 weeks following fMRI a subset of animals underwent behavior tests for anxiety and depression. (I) Sucrose preference test. Baseline measurements indicate no preference for either of the two bottles containing water. Test measurements with one bottle containing sucrose water and the other with water indicate both groups preferred the sucrose solution and no differences between the groups were observed (n=7.8, t-test, p>0.05). (J) Forced swim test. No differences were observed between groups following kindling (n=7.8, t-test, p>0.05). (K) Open field test. Kindled rats spent significantly less time in the center of the open field than in non-kindled rats indicating an increased anxiety phenotype following kindling (t-test, p=0.046). All data presented as mean±s.e.m. A threshold of p<0.05 was used for determination of statistical significance.

FIG. 3: Distinct propagation patterns in seizures induced in kindled and non-kindled rats. (A) Seizures were induced and assessed with simultaneous LFP-fMRI. Seizures were induced following a 90s baseline in non-kindled and kindled animals (n=2-4 per rat and n=7.5 rats respectively. (B) Seizure duration of the induced seizures. Seizures in kindled rats were significantly longer than those in the non-kindled rats (t-test, p<0.001). (C, D) Single seizure induced in a non-kindled rat with numbers indicating equivalent time points for LFP and BOLD maps. (C) LFP response overlaid with the ventral hippocampal BOLD response. (D) Brain-wide BOLD response to optogenetic seizure induction. Left panel: Brain slices corresponding to Paxinos rat brain atlas with coordinates relative to Bregma. Blue circle indicates site of seizure induction. Middle panel: evolution of BOLD activity during and after seizure induction. Right panel: voxel-level maximum intensity projection for experiment. Note that activity is mostly restricted to the ipsilateral hemisphere. (E, F) Single seizure induced in a kindled rat with numbers indicating equivalent time points for LFP and BOLD maps. (E) LFP response overlaid with the ventral hippocampal BOLD response. (F) Brain-wide BOLD response to optogenetic seizure induction. Left panel: Brain slices corresponding to Paxinos rat brain atlas with coordinates relative to Bregma. Blue circle indicates site of seizure induction. Middle panel: evolution of BOLD activity during and after seizure induction. Right panel: voxel-level maximum intensity projection for experiment. Note that activity propagates to bilaterally to cortex. BOLD images were normalized to baseline, defined as 60 s before stimulation onset. All images are displayed with a ±2% threshold for visualization.

FIG. 4. Kindled seizures gradually propagate to cortex bilaterally whereas non-kindled seizures remain localized. (A) Example of regional BOLD activity from a single optogenetically induced seizure. Regions were automatically segmented into 44 brain regions using a common brain atlas. Blue bar indicates optogenetic seizure induction period. (B) Number of regions activated in non-kindled (n=20 from 7 rats) and kindled seizures (n=17 from 5 rats). Seizures in the kindled rats activated 15.5±2.2 more regions than in non-kindled animals (p<0.0001). Grey circles indicate individual seizures. (C) Distribution of activated regions in non-kindled and kindled seizures. Each region from each group was normalized to the total number of seizures (n=20 and n=17, respectively) for comparison. Note that in non-kindled seizures, activated regions were mostly in the ipsilateral hemisphere in contrast to kindled seizures in which bilateral activation was more common. Black circle indicates an 80% threshold to ensure reliable onset time estimates for regional propagation analysis. (D) Regional propagation of activity in non-kindled and kindled seizures. Regions were sorted from fastest to slowest and white bars indicate ipsilateral regions and black bars contralateral regions for visualization. Left panel: In seizures in non-kindled rats, activated regions were most consistently observed in the ipsilateral hemisphere. Right panel: in contrast to seizures in kindled rats in which activity propagated to both hemispheres. Notably, activity propagated from ipsilateral to contralateral hemisphere. Red labels indicate regions activated in both two groups. (E) Onset time comparisons in commonly activated regions. iMePFC was activated significantly faster by 1.28±0.46 s in kindled seizures than in non-kindled seizures (t=2.153, p=0.044, corrected for multiple comparisons with Bonferroni procedure). A threshold of p<0.05 was used for determination of statistical significance.

FIG. 5: A slow migrating hippocampal seizure core occurs frequently in non-kindled and kindled rats, and can act as the primary mechanism for seizure generalization. (A,B) Single seizure induced in a non-kindled rat with numbers indicating equivalent time points for LFP and BOLD maps. (A) LFP response overlaid with the ventral hippocampal BOLD response. (B) Propagation of BOLD activity within ipsilateral hippocampus. Note that the high amplitude activity starts in the stimulated VHip and moves up the pole of the hippocampus to the dorsal region and that by timepoint 8, the activity is no longer detected in the VHip electrode but BOLD increases persist in DHip. (C,D) Single seizure induced in a kindled rat with numbers indicating corresponding time points for LFP and BOLD maps. (C) LFP response overlaid with the ventral hippocampal BOLD response. (D) Propagation of BOLD activity within ipsilateral hippocampus. Similar to (A, B), BOLD and LFP increases in vHip during seizure induction and continues to increase. High amplitude activity moves from VHip (time points 3,4) and then up the pole to DHip (time points 5,6). (E) Segmentation atlas of ipsilateral hippocampus. Hippocampus was segmented into 4 regions across 10 slices. (F) Activity propagation time from ventral hippocampus to dorsal hippocampus. Peak to peak activity from most ventral to most dorsal of the segmented regions were used to calculate the time that peak activity was observed in the ventral to dorsal hippocampus. Propagating hippocampal activity was observed in 8/20 seizures in 4/7 non-kindled rats and 15/17 seizures in 5/5 kindled rats (G, H) Individual hippocampal voxel time courses in non-kindled and kindled animal. Time courses are from the same seizures in (A, C) and segmented as in (E). Voxel time courses are sorted from the most ventral to most dorsal.

FIG. 6: Voxel-wise between group differences only apparent after kindling and not before. Statistical t-maps after kindling (right panel) and in cage-mate age-matched controls (left panel) with a t-threshold corresponding to p<0.001.

FIG. 7: Number of stimulations or stage 5 seizures did not explain post-kindling activation. Statistical voxel-wise t-maps of post-kindling activation vs number of stimulations (left panel) and vs number of stage 5 seizures (right panel) with a t-threshold corresponding to p<0.001.

FIG. 8: Increased amplitude of medial prefrontal response to ventral hippocampal 10 Hz stimulation following kindling. Regional time series data from the ventral hippocampal circuit.

FIG. 9: Simultaneous LFP recordings that resulted in removal from analysis. (A-C) top two panels are LFP recordings acquired simultaneously with fMRI from the ventral hippocampus and the ipsilateral medial prefrontal cortex. Lower two panels are a magnification of those recordings. Blue bar indicates optogenetic stimulation (10 Hz with 7.5 ms pulses). (A) Bursting following offset of stimulation. Animal was removed from electrophysiological and BOLD analyses. (B,C) Medial prefrontal cortical electrode was broken resulting in noise recordings. Both animals were removed from analysis of the medial prefrontal cortical LFP.

FIG. 10: Similar electrophysiological characteristics in seizures induced in awake and dexmedetomidine-sedated rats. (A,B) Ventral hippocampal LFP recordings from the same animal and seizures induced when awake and under sedation. Red arrow indicates large amplitude spike onset, and insets i-iii) are magnified components of those seizures. Blue bar indicates time of optogenetic stimulation (40 Hz with 7.5 ms pulses) (C) Another set of electrophysiological recordings from a different animal in the awake and sedated state. (D) No differences in the proportion of stimulations resulting is afterdischarges in awake and sedated states. Each line represents a single animal. (E) No differences in the large amplitude spike onset in awake and sedated states. (F) Sedation did not result in shorter afterdischarges in sedated states compared to the awake state.

FIG. 11: Regional propagation of activity in non-kindled and kindled seizures using a 90% onset. Regions were sorted from fastest to slowest and white bars indicate ipsilateral regions and black bars contralateral regions for visualization. Left panel: In seizures in non-kindled rats, activated regions were most consistently observed in the ipsilateral hemisphere. Right panel: in contrast to seizures in kindled rats in which activity propagated to both hemispheres. Notably, activity propagated from ipsilateral to contralateral hemisphere.

FIG. 12: Regional propagation of activity in non-kindled and kindled seizures using a 0% onset frequency. Regions were sorted from fastest to slowest and white bars indicate ipsilateral regions and black bars contralateral regions for visualization.

FIG. 13: Distinct regional cross correlation patterns during seizure in non-kindled and kindled animals. Top panel: Averaged regional cross correlation matrix in non-kindled animals. Lower panel: Mean regional cross correlation matrix in kindled animals.

### DETAILED DESCRIPTION

### Definitions

Before embodiments of the present disclosure are further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Any methods and materials similar or equivalent to those described herein can also be used in the practice or testing of embodiments of the present disclosure.

It must be noted that as used herein and in the appended claims, the singular forms “a”, “and”, and “the” include plural referents unless the context clearly dictates otherwise. Thus, for example, reference to “a compound” includes not only a single compound but also a combination of two or more compounds, reference to “a substituent” includes a single substituent as well as two or more substituents, and the like.

In describing and claiming the present invention, certain terminology will be used in accordance with the definitions set out below. It will be appreciated that the definitions provided herein are not intended to be mutually exclusive. Accordingly, some chemical moieties may fall within the definition of more than one term.

As used herein, the phrases “for example,” “for instance,” “such as,” or “including” are meant to introduce examples that further clarify more general subject matter. These examples are provided only as an aid for understanding the disclosure, and are not meant to be limiting in any fashion.

The terms “active agent”, “antagonist”, “inhibitor”, “drug” and “pharmacologically active agent” are used interchangeably herein to refer to a chemical material or compound which, when administered to an organism (human or animal) induces a desired pharmacologic and/or physiologic effect by local and/or systemic action.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease.

A “therapeutically effective amount” or “efficacious amount” means the amount of a compound that, when administered to a mammal or other subject for treating a disease, condition, or disorder, is sufficient to effect such treatment for the disease, condition, or disorder. The “therapeutically effective amount” will vary depending on the compound, the disease and its severity and the age, weight, etc., of the subject to be treated.

The term “unit dosage form,” as used herein, refers to physically discrete units suitable as unitary dosages for human and animal subjects, each unit containing a predetermined quantity of a compound calculated in an amount sufficient to produce the desired effect in association with a pharmaceutically acceptable diluent, carrier or vehicle. The specifications for unit dosage forms depend on the particular compound employed and the effect to be achieved, and the pharmacodynamics associated with each compound in the host.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, carrier, and adjuvant.

As used herein, a “pharmaceutical composition” is meant to encompass a composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal, intramuscular, subcutaneous, and the like.

The terms “individual,” “host,” “subject,” and “patient” are used interchangeably herein, and refer to an animal, including, but not limited to, human and non-human primates, including simians and humans; rodents, including rats and mice; bovines; equines; ovines; felines; canines; avians, and the like. “Mammal” means a member or members of any mammalian species, and includes, by way of example, canines; felines; equines; bovines; ovines; rodentia, etc. and primates, e.g., non-human primates, and humans. Non-human animal models, e.g., mammals, e.g. non-human primates, murines, lagomorpha, etc. may be used for experimental investigations. Suitable animal models include particularly rodents, e.g. rats and mice.

As used herein, the terms “determining,” “measuring,” “assessing,” and “assaying” are used interchangeably and include both quantitative and qualitative determinations.

Local Field Potential (LFP) is the electric potential recorded in the extracellular space in brain tissue, typically using micro-electrodes (metal, silicon or glass micropipettes). LFPs are recorded in depth, from within the cortical tissue (or other deep brain structures). The LFP signal in the mammalian cortex reflects the activity of thousands of neurons and is commonly used to study the network dynamics underlying e.g., sensory processing, motor planning, attention, memory, and perception. The LFP signal has further increased in importance in recent decades because of the development of high-density silicon-based microelectrodes, allowing simultaneous recording of the LFP at thousands of positions spanning entire brain regions. LFP can be used for steering neuroprosthetic devices as it is easier and more stably recorded in chronic settings than single-unit spiking activity.

Magnetic resonance imaging (MRI) is used to analyze neurophysical events. In particular, MRI can be used to analyze functionally correlated regions of the brain (anatomical neural networks) in relation to neurophysical events. The correlation patterns can denote a temporal and/or spatial correlation of neurophysical events. An MRI technique of interest is functional MRI (fMRI). With fMRI, temporal changes in image contrast are displayed by suitable MR imaging scanning sequences. Functional MRI (fMRI) measures signal changes in the brain that are due to changing neural activity. The brain is scanned at low resolution but at a rapid rate (typically once every 2-3 seconds). Increases in neural activity cause changes in the MR signal via T*.sub.2 changes. This mechanism is referred to as the blood-oxygen-level dependent (BOLD) effect. Increased neural activity causes an increased demand for oxygen, and the vascular system actually overcompensates for this, increasing the amount of oxygenated hemoglobin relative to deoxygenated hemoglobin. Because deoxygenated hemoglobin attenuates the MR signal, the vascular response leads to a signal increase that is related to the neural activity. The BOLD effect also allows for the generation of high resolution 3D maps of the venous vasculature within neural tissue.

While a BOLD signal is the most common method employed for neuroscience studies in human subjects, the flexible nature of MR imaging provides means to sensitize the signal to other aspects of the blood supply. Alternative techniques employ arterial spin labeling (ASL) or weight the MRI signal by cerebral blood flow (CBF) and cerebral blood volume (CBV). The CBV method requires injection of a class of MRI contrast agents that are now in human clinical trials. Because this method has been shown to be far more sensitive than the BOLD technique in preclinical studies, it may potentially expand the role of fMRI in clinical applications. The CBF method provides more quantitative information than the BOLD signal, albeit at a significant loss of detection sensitivity.

Epilepsy. Epilepsy is a brain disorder characterized by repeated seizures over time. Types of epilepsy can include, but are not limited to generalized epilepsy, e.g., childhood absence epilepsy, juvenile myoclonic epilepsy, epilepsy with grand-mal seizures on awakening, West syndrome, Lennox-Gastaut syndrome, partial epilepsy, e.g., temporal lobe epilepsy, frontal lobe epilepsy, benign focal epilepsy of childhood.

Status Epilepticus (SE). Status epilepticus (SE) can include, e.g., convulsive status epilepticus, e.g., early status epilepticus, established status epilepticus, refractory status epilepticus, super-refractory status epilepticus; non-convulsive status epilepticus, e.g., generalized status epilepticus, complex partial status epilepticus; generalized periodic epileptiform discharges; and periodic lateralized epileptiform discharges. Convulsive status epilepticus is characterized by the presence of convulsive status epileptic seizures, and can include early status epilepticus, established status epilepticus, refractory status epilepticus, super-refractory status epilepticus. Early status epilepticus is treated with a first line therapy. Established status epilepticus is characterized by status epileptic seizures which persist despite treatment with a first line therapy, and a second line therapy is administered. Refractory status epilepticus is characterized by status epileptic seizures which persist despite treatment with a first line and a second line therapy, and a general anesthetic is generally administered. Super refractory status epilepticus is characterized by status epileptic seizures which persist despite treatment with a first line therapy, a second line therapy, and a general anesthetic for 24 hours or more.

Non-convulsive status epilepticus can include, e.g., focal non-convulsive status epilepticus, e.g., complex partial non-convulsive status epilepticus, simple partial non-convulsive status epilepticus, subtle non-convulsive status epilepticus; generalized non-convulsive status epilepticus, e.g., late onset absence non-convulsive status epilepticus, atypical absence non-convulsive status epilepticus, or typical absence non-convulsive status epilepticus.

Seizure. A seizure is the physical findings or changes in behavior that occur after an episode of abnormal electrical activity in the brain. The term “seizure” is often used interchangeably with “convulsion.” Convulsions are when a person's body shakes rapidly and uncontrollably. During convulsions, the person's muscles contract and relax repeatedly. Based on the type of behavior and brain activity, seizures are divided into two broad categories: generalized and partial (also called local or focal). Classifying the type of seizure helps doctors diagnose whether or not a patient has epilepsy.

Generalized seizures are produced by electrical impulses from throughout the entire brain, whereas partial seizures are produced (at least initially) by electrical impulses in a relatively small part of the brain. The part of the brain generating the seizures is sometimes called the focus.

There are a number of types of generalized seizures. The most common and dramatic, and therefore the most well known, is the generalized convulsion, also called the grand-mal seizure. In this type of seizure, the patient loses consciousness and usually collapses. The loss of consciousness is followed by generalized body stiffening (called the “tonic” phase of the seizure) for 30 to 60 seconds, then by violent jerking (the “clonic” phase) for 30 to 60 seconds, after which the patient goes into a deep sleep (the “postictal” or after-seizure phase). During grand-mal seizures, injuries and accidents may occur, such as tongue biting and urinary incontinence.

Absence seizures cause a short loss of consciousness (just a few seconds) with few or no symptoms. The patient, most often a child, typically interrupts an activity and stares blankly. These seizures begin and end abruptly and may occur several times a day. Patients are usually not aware that they are having a seizure, except that they may be aware of “losing time.”

Myoclonic seizures consist of sporadic jerks, usually on both sides of the body. Patients sometimes describe the jerks as brief electrical shocks. When violent, these seizures may result in dropping or involuntarily throwing objects.

Clonic seizures are repetitive, rhythmic jerks that involve both sides of the body at the same time.

Tonic seizures are characterized by stiffening of the muscles.

Atonic seizures consist of a sudden and general loss of muscle tone, particularly in the arms and legs, which often results in a fall.

Focal to bilateral tonic-clonic (FBTC) seizures start in one area of the brain, then spreads to both sides of the brain as a tonic-clonic seizure.

When assessing seizure severity, a scale is often used to categorize seizure-related behaviors. The Racine scale has been the scale most widely used to describe these behaviors. The Racine scale comprises 5 stages and each stage is categorized as follows: Stage 1: mouth and facial clonus; Stage 2: Stage 1+head nodding; Stage 3: Stage 2+forelimb clonus; Stage 4: Stage 3+rearing; Stage 5: Stage 4+repeated rearing and falling.

As used herein, the term “kindling” or “kindling model” refers to the widely used model for the development of seizures and epilepsy, in which the duration and behavioral involvement of induced seizures increases after seizures are induced repeatedly. In such models, experimental animals are repeatedly stimulated, usually with electricity or chemicals, to induce the seizures. The seizure that occurs after the first such stimulation lasts a short time and is accompanied by a small amount or no behavioral effects compared with the seizures that result from repeated stimulations. With further seizures, the accompanying behavior intensifies, for example, progressing from a freezing in early stimulations to convulsions in later ones. The lengthening of duration and intensification of behavioral accompaniment eventually reaches a plateau after repeated stimulations (see, for example, Bertram, E., (2007) Epilepsia 48 (Supplement 2): 65-74).

Kindling can be achieved using multiple methods, including but not limited to, electrostimulation, optogenetics, chemical treatment, etc. When optogenetics is used for kindling a light-activatable protein is expressed in target cells of interest. Light-activatable proteins that may be used, include but are not limited to, ChR2, VChR1, C1V1, etc. In some embodiments, expression of the light-activatable protein is targeted to neurons of interest using the Ca2+/calmodulin-dependent protein kinase II (CaMKII) promoter. In some embodiments, a polynucleotide encoding the light-activatable protein is delivered to the hippocampus.

Animals may be stimulated up to a maximum of 12 stimulations a day or until the emergence of a stage 5 motor seizure and stimulated every other day up to a maximum of 12 days of stimulations. In some embodiments, animals are stimulated less than 12 times per day, for example, 11 to 9 times, 9 to 7 times, 7 to 5 times, 5 to 3 times, or less than 3 times a day until kindling is achieved. Stimulations may be carried out for a maximum of 12 days. In some embodiments, the animal is stimulated for 11, 10, 9, 8, 7, 6, 5, 4, 3, 2, or 1 day to achieve kindling. Interstimulus interval may be 15 minutes. Individuals may be considered to be kindled if a stage 5 motor seizure was observed within the first three stimulations of the day.

Seizure onset zone. The seizure onset zone is the area of the cortex from which clinical seizures are generated, as opposed to the epileptogenic zone, which is the area of the cortex that is indispensable for the generation of epileptic seizures. The seizure onset zone is commonly localized by either scalp or invasive EEG techniques. The location of the seizure onset zone can also be determined by ictal single photon emission computed tomography (SPECT). It is usually the portion of the irritative zone that generates spikes capable of producing afterdischarges. These consist of repetitive spikes that have enough strength to produce clinical ictal symptoms when invading eloquent cortex. Invasive cortical surface electrodes record activity from an extremely limited region of the brain. By eliminating distance and the insulating barriers, each electrode records the cortical area covered by only that electrode. Invasive electrodes are, therefore, inherently very sensitive for the detection of afterdischarges but will only be able to define the seizure origin accurately if they cover the seizure onset zone directly.

“Neural activity” as used herein, may refer to electrical activity of a neuron (e.g., changes in membrane potential of the neuron), as well as indirect measures of the electrical activity of one or more neurons. Thus, neural activity may refer to changes in field potential, changes in intracellular ion concentration (e.g., intracellular calcium concentration), and changes in magnetic resonance induced by electrical activity of neurons, as measured by, e.g., blood oxygenation level dependent (BOLD) signals in functional magnetic resonance imaging.

### Seizure Models

Provided herein are methods and models for analyzing in vivo the brain circuits and regional relationships involved in seizures, particularly by imaging of single seizures for fine discrimination of effects. Methods of the present disclosure may use any number of combinations of suitable neuronal stimulation and neuronal activity measurement protocols, as necessary, to image the effects of seizures. The methods and models may include, for example, one or more of analysis of single seizures, analysis of focal to bilateral tonic-clonic (FBTC) seizures, analysis of excitatory ventral hippocampal (VH) networks, and the like.

The models provided herein generally use kindled animal models. Kindling refers to a seizure-induced plasticity phenomenon that occurs when repeated after discharge induction by electrical stimulation in a specific brain region evokes a progressive enhancement of seizure susceptibility. Ultimately, it culminates in emergence of spontaneous seizures and the establishment of a permanent epileptic state. Kindling can be established with optogenetic or electrical stimulation.

To image single seizures with simultaneous LFP-fMRI in an animal model, the animal may be sedated and treated with a short-acting neuromuscular blocker to abolish motion during imaging of seizures, exemplary agents for this purpose include, without limitation, dexmedetomidine sedation and vecuronium. Through imaging of individual seizures a core of slow migrating activity in the hippocampus is shown to provide a novel seizure propagation and generalization mechanism; and the propagation of FBTC seizures can be imaged.

Seizures may be induced in the kindled animal with optogenetic stimulation, with electrical stimulation, e.g. electroshock whole-brain stimulation protocols, single-evoked epileptic afterdischarges; with chemoconvulsants, e.g. pilocarpine, tetanus toxin, PTZ, kainic acid, flurothyl, etc., fluid percussion injury, high-intensity acoustic stimulation. In some embodiments optogenetic stimulation is preferred.

In one embodiment a specific region of a brain of an individual is stimulated, in conjunction with combined electrophysiology, e.g. local field potentials (LFP) and functional magnetic resonance imaging (fMRI) scanning of different regions of the brain to determine functional connections between the seizure propagation zone and other regions of the brain and to image movement of a seizure. Suitable protocols for analysis include electrophysiology; light-induced modulation of neural activity; electroencephalography (EEG) recordings; functional imaging and behavioral analysis. Electrophysiology may include single electrode, multi electrode, and/or field potential recordings. Light-induced modulation of neural activity may include any suitable optogenetic method, as described further herein. Functional imaging may include fMRI, and any functional imaging protocols using genetically encoded indicators (e.g., calcium indicators, voltage indicators, etc.). Behavioral analysis may include any suitable behavioral assays, such as behavioral assays for arousal, memory (such as a water maze assay), conditioning (such as fear conditioning), sensory responses (responses to e.g., visual, somatosensory, auditory, gustatory, and/or olfactory cues).

Some protocols, such as fMRI, provide a non-invasive, brain-wide measure representative of neural activity. Some protocols, such as electrophysiology, provide cellular resolution and rapid measures of neural activity as well as cellular resolution and rapid control of neural activity. Some protocols, such as optogenetics, provide spatially-targeted and temporally-defined control of action potential firing in defined groups of neurons.

In some embodiments, methods are provided for specific identification and localization of migrating seizure cores in an individual by induction and propagation from a single seizure in a kindled animal, which may find use in determining a seizure onset zone. In some embodiments methods are provided for specific identification and localization of FBTC seizures in an individual by induction and propagation from a single seizure in a kindled animal.

In some embodiments an optogenetic kindling model for seizures is provided, wherein electrographic seizures are induced in an animal model by cell-type specific, optogenetic stimulations, which animal then provides for reliable induction of FBTC seizures over an extended period of time, where an extended period of time may be up to two weeks, up to 3 weeks, up to 4 weeks, up to 2 months, up to 3 months, or more. The light-activated polypeptide for stimulation may be, for example, a channelrhodopsin, including without limitation CHR2. The light activated polypeptide may be operably linked to a promoter expressed in excitatory hippocampal neurons. Stimulation paradigms include a series of short and mild stimulations below the threshold to trigger seizures, e.g. of around about 10 Hz, to evaluate underlying functional circuit changes. A long and intense stimulation of about 40 Hz can be used to evaluate seizure circuit dynamics.

In some embodiments an optogenetic kindling model for seizures is provided, wherein electrographic seizures are induced in an animal model by cell-type specific, optogenetic stimulations, which animal then provides for reliable induction of FBTC seizures over an extended period of time, where an extended period of time may be up to two weeks, up to 3 weeks, up to 4 weeks, up to 2 months, up to 3 months, or more. Stimulation paradigms include a series of short and mild stimulations below the threshold to trigger seizures, e.g. of around about 8-12 Hz, e.g. about 10 Hz, to evaluate underlying functional circuit changes. A long and intense stimulation of about 35-45 Hz, e.g. about 40 Hz can be used to evaluate seizure circuit dynamics. Simultaneous electrophysiology and fMRI can be used to determine the effect of kindling excitatory neurons in the ventral hippocampus. Imaging brain-wide network dynamics of single induced seizures shows focal and FBTC seizure propagation. In some embodiments these variables of seizure propagation are used to guide surgical targeting and therapy development for epilepsy. Included in the findings is the showing of a slow migrating core of high amplitude activity that can accompany seizures, and is frequently observed prior to seizures. The animal model is useful in the design and testing of therapeutic interventions, e.g. surgery, pharmacologic therapy, and the like, where the effect of a therapeutic intervention on seizure propagation can be determined. The animal model is also useful in the design of drugs for epilepsy co-morbidities, e.g. the largest activity changes are in the medial prefrontal cortex (mePFC), indicating an increased excitatory relationship between ventral hippocampus (vHip) and mePFC. Therapies designed to target vHip-mePFC circuit dysfunction can reduce comorbidities associated with epilepsy, including anxiety and cognitive defects.

In some embodiments, the animal model is kindled. Kindling may be achieved using multiple methods, including but not limited to, electrostimulation, optogenetics, chemical treatment, etc. When optogenetics is used for kindling, a light-activatable protein may be expressed in target cells of interest. Light-activatable proteins that may be used, include but are not limited to, ChR2, VChR1, C1V1, etc. In some embodiments, the light-activatable protein is targeted to neurons of interest using the Ca2+/calmodulin-dependent protein kinase II (CaMKII) promoter. In some embodiments, the polynucleotide encoding the light-activatable protein is delivered to the hippocampus.

To achieve kindling, the individual may be stimulated by at least 30 Hz of light with a pulse width of 7.5 ms. In some embodiments, the individual is stimulated with greater than 30 Hz. For example, 30-35 Hz, 35-40 Hz, 40-45 Hz, 45-50 Hz or greater than 50 Hz. To achieve kindling, the individual may be stimulated up to a maximum of 12 times per day. In some embodiments, subjects may be stimulated less than 12 times per day, for example, 11 to 9 times, 9 to 7 times, 7 to 5 times, 5 to 3 times, or less than 3 times a day until kindling is achieved. Stimulations may be carried out for a maximum of 12 days. In some embodiments, the individual may by stimulated for 11, 10, 9, 8, 7, 6, 5, 4, 3, 2, or 1 day.

To investigate functional neural circuit changes, an individual may be stimulated with at least 1 Hz of light pulses. In some embodiments, the individual may be stimulated with more than 1 Hz light pulses such as 1-5, 5-10, 10-15, or 15-20 Hz.

To investigate seizure neural circuit changes, an individual may be stimulated with at least 30 Hz of light pulses. In some embodiments, the individual may be stimulated with more than 30 Hz light pulses such as 30-35, 35-40, 40-45, 45-50 or greater than 50 Hz.

In some embodiments, an agent is determined to be effective for targeted intervention of seizures if the duration or severity of seizure is reduced. In some embodiments, the severity of a seizure is determined using the Racine scale. In some embodiments, an agent is determined to be effective for targeted intervention if seizure severity is reduced by at least 1 stage on the Racine scale. For example, reducing seizure severity from Racine stage 5 to stage 4. In some embodiments, seizure severity is reduced by 2 stages, 3 stages, 4 stages or seizures are stopped all together.

### Optogenetic Stimulation

As described above, animal models useful for detection of single seizures and the electrophysiology that derives therefrom may utilize optogenetic stimulation, e.g. for kindling, and for seizure induction, where the neurons involved in kindling and in seizure induction operably express a light-activated polypeptide.

The light stimulus used to activate the light-activated polypeptide may include light pulses characterized by, e.g., frequency, pulse width, duty cycle, wavelength, intensity, etc. In some cases, the light stimulus includes two or more different sets of light pulses, where each set of light pulses is characterized by different temporal patterns of light pulses. The temporal pattern may be characterized by any suitable parameter, including, but not limited to, frequency, period (i.e., total duration of the light stimulus), pulse width, duty cycle, etc.

The light pulses may have any suitable frequency. In some cases, the set of light pulses contains a single pulse of light that is sustained throughout the duration of the light stimulus. In some cases, the light pulses of a set have a frequency of 0.1 Hz or more, e.g., 0.5 Hz or more, 1 Hz or more, 5 Hz or more, 10 Hz or more, 20 Hz or more, 30 Hz or more, 40 H or more, including 50 Hz or more, or 60 Hz or more, or 70 Hz or more, or 80 Hz or more, or 90 Hz or more, or 100 Hz or more, and have a frequency of 100,000 Hz or less, e.g., 10,000 Hz or less, 1,000 Hz or less, 500 Hz or less, 400 Hz or less, 300 Hz or less, 200 Hz or less, including 100 Hz or less. In some embodiments, the light pulses have a frequency in the range of 0.1 to 100,000 Hz, e.g., 1 to 10,000 Hz, 1 to 1,000 Hz, including 5 to 500 Hz, or 10 to 100 Hz.

For example, a series of short and mild stimulations below the threshold to trigger seizures may be delivered, e.g. of around about 1 Hz to about 15 Hz, from about 5 Hz to about 15 Hz, and may be around 10 Hz can be applied to evaluate underlying functional circuit changes. A long and intense stimulation may be delivered, of from about 25 to about 50 Hz, of from about 35 to about 45 Hz, for example around about 40 Hz can be used to evaluate seizure circuit dynamics.

In some cases, the two sets of light pulses are characterized by having different parameter values, such as different pulse widths, e.g. short or long. The light pulses may have any suitable pulse width. In some cases, the pulse width is 0.1 ms or longer, e.g., 0.5 ms or longer, 1 ms or longer, 3 ms or longer, 5 ms or longer, 7.5 ms or longer, 10 ms or longer, including 15 ms or longer, or 20 ms or longer, or 25 ms or longer, or 30 ms or longer, or 35 ms or longer, or 40 ms or longer, or 45 ms or longer, or 50 ms or longer, and is 500 ms or shorter, e.g., 100 ms or shorter, 90 ms or shorter, 80 ms or shorter, 70 ms or shorter, 60 ms or shorter, 50 ms or shorter, 45 ms or shorter, 40 ms or shorter, 35 ms or shorter, 30 ms or shorter, 25 ms or shorter, including 20 ms or shorter. In some embodiments, the pulse width is in the range of 0.1 to 500 ms, e.g., 0.5 to 100 ms, 1 to 80 ms, including 1 to 60 ms, or 1 to 50 ms, or 1 to 30 ms.

The average power of the light pulse, measured at the tip of an optical fiber delivering the light pulse to regions of the brain, may be any suitable power. In some cases, the power is 0.1 mW or more, e.g., 0.5 mW or more, 1 mW or more, 1.5 mW or more, including 2 mW or more, or 2.5 mW or more, or 3 mW or more, or 3.5 mW or more, or 4 mW or more, or 4.5 mW or more, or 5 mW or more, and may be 1,000 mW or less, e.g., 500 mW or less, 250 mW or less, 100 mW or less, 50 mW or less, 40 mW or less, 30 mW or less, 20 mW or less, 15 mW or less, including 10 mW or less, or 5 mW or less. In some embodiments, the power is in the range of 0.1 to 1,000 mW, e.g., 0.5 to 100 mW, 0.5 to 50 mW, 1 to 20 mW, including 1 to 10 mW, or 1 to 5 mW.

The wavelength and intensity of the light pulses may vary and may depend on the activation wavelength of the light-activated polypeptide, optical transparency of the region of the brain, the desired volume of the brain to be illuminated, etc.

The volume of a brain region illuminated by the light pulses may be any suitable volume. In some cases, the illuminated volume is 0.001 mm³ or more, e.g., 0.005 mm³ or more, 0.001 mm³ or more, 0.005 mm³ or more, 0.01 mm³ or more, 0.05 mm³ or more, including 0.1 mm³ or more, and is 100 mm³ or less, e.g., 50 mm³ or less, 20 mm³ or less, 10 mm³ or less, 5 mm³ or less, 1 mm³ or less, including 0.1 mm³ or less. In certain cases, the illuminated volume is in the range of 0.001 to 100 mm³, e.g., 0.005 to 20 mm³, 0.01 to 10 mm³, 0.01 to 5 mm³, including 0.05 to 1 mm³.

Optogenetic stimulation can be performed using any suitable method. Suitable methods are described in, e.g., U.S. Pat. No. 8,834,546, which is incorporated herein by reference. Neurons of a suitable region of the brain whose activity is to be modulated by light can be modified using a convenient method to express the light-activated polypeptide. In some cases, neurons of a brain region are genetically modified to express a light-activated polypeptide. In some cases, the neurons may be genetically modified using a viral vector, e.g., an adeno-associated viral vector, containing a nucleic acid having a nucleotide sequence that encodes the light-activated polypeptide. The viral vector may include any suitable control elements (e.g., promoters, enhancers, recombination sites, etc.) to control expression of the light-activated polypeptide according to cell type, timing, presence of an inducer, etc. In some cases, cell type-specific expression of the light-activated polypeptide may be achieved by using recombination systems, e.g., Cre-Lox recombination, Flp-FRT recombination, etc. Cell type-specific expression of genes using recombination has been described in, e.g., Fenno et al., Nat Methods. 2014 July; 11(7):763; and Gompf et al., Front Behav Neurosci. 2015 Jul. 2; 9:152, which are incorporated by reference herein.

Suitable neuron-specific control sequences include, without limitation, an alpha subunit of Ca⁺⁺-calmodulin-dependent protein kinase II (CaMKIIα) promoter (see, e.g., Mayford et al. (1996) Proc. Natl. Acad. Sci. USA 93:13250) to target ventral hippocampal CAMKII neurons.

In some cases the regions of the brain with neurons containing a light-activated peptide is illuminated using one or more optical fibers. The optical fiber may be configured in any suitable manner to direct a light emitted from suitable source of light, e.g., a laser or light-emitting diode (LED) light source, to the region of the brain. The optical fiber may be any suitable optical fiber. In some cases, the optical fiber is a multimode optical fiber. The optical fiber may include a core defining a core diameter, where light from the light source passes through the core. The optical fiber may have any suitable core diameter. In some cases, the core diameter of the optical fiber is 10 μm or more, e.g., 20 μm or more, 30 μm or more, 40 μm or more, 50 μm or more, 60 μm or more, including 80 μm or more, and is 1,000 μm or less, e.g., 500 μm or less, 200 μm or less, 100 μm or less, including 70 μm or less. In some embodiments, the core diameter of the optical fiber is in the range of 10 to 1,000 μm, e.g., 20 to 500 μm, 30 to 200 μm, including 40 to 100 μm.

The optical fiber end that is implanted into the target region of the brain may have any suitable configuration suitable for illuminating a region of the brain with a light stimulus delivered through the optical fiber. In some cases, the optical fiber includes an attachment device at or near the distal end of the optical fiber, where the distal end of the optical fiber corresponds to the end inserted into the subject. In some cases, the attachment device is configured to connect to the optical fiber and facilitate attachment of the optical fiber to the subject, such as to the skull of the subject. Any suitable attachment device may be used. In some cases, the attachment device includes a ferrule, e.g., a metal, ceramic or plastic ferrule. The ferrule may have any suitable dimensions for holding and attaching the optical fiber.

In certain embodiments, methods of the present disclosure may be performed using any suitable electronic components to control and/or coordinate the various optical components used to illuminate the regions of the brain. The optical components (e.g., light source, optical fiber, lens, objective, mirror, and the like) may be controlled by a controller, e.g., to coordinate the light source illuminating the regions of the brain with light pulses. The controller may include a driver for the light source that controls one or more parameters associated with the light pulses, such as, but not limited to the frequency, pulse width, duty cycle, wavelength, intensity, etc. of the light pulses. The controllers may be in communication with components of the light source (e.g., collimators, shutters, filter wheels, movable mirrors, lenses, etc.).

A number of light-activated polypeptides are known in the art for optogenetic use, including, for example, a light-activated ion channel or a light-activated ion pump. See, for example, Repina et al. Annu Rev Chem Biomol Eng. 2017 Jun. 7; 8: 13-39; WO2014144409A1; U.S. Pat. Nos. 10,220,092; 10,371,776; PCT/US2011/028893; WO/2013/093463; WO/2017/210664; WO/2017/015395; WO/2019/092564; WO/2017/100058, each herein specifically incorporated by reference. The light-activated polypeptides are activated by different wavelengths of light, including, for example, blue light; green light; yellow light; orange light; red light. The light activated polypeptide can be fused to various sequences, e.g. signal peptides, an endoplasmic reticulum (ER) export signal, a membrane trafficking signal, and/or an N-terminal golgi export signal, etc.; including addition of a trafficking signal (ts) that enhances transport of the protein to the cell plasma membrane.

Light-activated polypeptides of interest include, for example, a step function opsin (SFO)6 protein or a stabilized step function opsin (SSFO) protein that can have specific amino acid substitutions at key positions in the retinal binding pocket of the protein. See, for example, WO 2010/056970, the disclosure of which is hereby incorporated by reference in its entirety. The polypeptide may be a cation channel derived from Volvox carteri (VChR1), optionally comprising one or more amino acid substitutions, e.g. C123A; C123S; D151A, etc. A light-activated cation channel protein can be a C1V1 chimeric protein derived from the VChR1 protein of Volvox carteri and the ChR1 protein from Chlamydomonas reinhardti, wherein the protein comprises the amino acid sequence of VChR1 having at least the first and second transmembrane helices replaced by the first and second transmembrane helices of ChR1, optionally having an amino acid substitution at amino acid residue E122 or E162. In other embodiments, the light-activated cation channel protein is a C1C2 chimeric protein derived from the ChR1 and the ChR2 proteins from Chlamydomonas reinhardti, wherein the protein is responsive to light and is capable of mediating a depolarizing current in the cell when the cell is illuminated with light. In some embodiments, a depolarizing light-activated polypeptide is a red shifted variant of a depolarizing light-activated polypeptide derived from Chlamydomonas reinhardtii; referred to as a “ReaChR polypeptide” or “ReaChR protein” or “ReaChR.” In some embodiments, a depolarizing light-activated polypeptide is a SdChR polypeptide derived from Scherffelia dubia, wherein the SdChR polypeptide is capable of transporting cations across a cell membrane when the cell is illuminated with light. In some embodiments, a depolarizing light-activated polypeptide is CnChR1, derived from Chlamydomonas noctigama, wherein the CnChR1 polypeptide is capable of transporting cations across a cell membrane when the cell is illuminated with light. In some embodiments, the light-activated cation channel protein is a CsChrimson chimeric protein derived from a CsChR protein of Chloromonas subdivisa and CnChR1 protein from Chlamydomonas noctigama, wherein the N terminus of the protein comprises the amino acid sequence of residues 1-73 of CsChR followed by residues 79-350 of the amino acid sequence of CnChR1; is responsive to light; and is capable of mediating a depolarizing current in the cell when the cell is illuminated with light. In some embodiments, a depolarizing light-activated polypeptide can be, e.g. ShChR1, derived from Stigeoclonium helveticum, wherein the ShChR1 polypeptide is capable of transporting cations across a cell membrane when the cell is illuminated with light.

In some embodiments, a depolarizing light-activated polypeptide is derived from Chlamydomonas reinhardtii (CHR1, and particularly CHR2) wherein the polypeptide is capable of transporting cations across a cell membrane when the cell is illuminated with light; and is capable of mediating a depolarizing current in the cell when the cell is illuminated with light. In some embodiments CaMKIIa-driven, humanized channelrhodopsin CHR2 H134R mutant fused to EYFP is used for optogenetic activation. The light used to activate the light-activated cation channel protein derived from Chlamydomonas reinhardtii can have a wavelength between about 460 and about 495 nm or can have a wavelength of about 480 nm. The light-activated cation channel protein can additionally comprise substitutions, deletions, and/or insertions introduced into a native amino acid sequence to increase or decrease sensitivity to light, increase or decrease sensitivity to particular wavelengths of light, and/or increase or decrease the ability of the light-activated cation channel protein to regulate the polarization state of the plasma membrane of the cell. Additionally, the light-activated cation channel protein can comprise one or more conservative amino acid substitutions and/or one or more non-conservative amino acid substitutions. The light-activated proton pump protein containing substitutions, deletions, and/or insertions introduced into the native amino acid sequence suitably retains the ability to transport cations across a cell membrane. The protein may comprise various amino acid substitutions, e.g. one or more of H134R; T159C; L132C; E123A; etc. The protein may further comprise a fluorescent protein, for example, but not limited to, a yellow fluorescent protein, a red fluorescent protein, a green fluorescent protein, or a cyan fluorescent protein.

### Drug Design

Methods are provided for optimizing therapy, by analyzing the effects of seizures across brain regions, and based on that information, selecting appropriate drug candidates and therapeutic modalities that are optimal for addressing seizure induction and propagation, while minimizing undesirable toxicity. The treatment is optimized by selection for a treatment that minimizes undesirable toxicity, while providing for effective activity.

The models provided herein are useful in the design and testing of therapeutic interventions, e.g. surgery, pharmacologic therapy, and the like, where the effect of a therapeutic intervention on seizure induction and propagation can be determined. The models are also useful in the design of drugs for epilepsy co-morbidities, e.g. the largest activity changes are in the medial prefrontal cortex (mePFC), indicating an increased excitatory relationship between ventral hippocampus (vHip) and mePFC. Therapies designed to target vHip-mePFC circuit dysfunction can reduce comorbidities associated with epilepsy, including anxiety and cognitive defects. In some embodiments these variables of seizure propagation are used to guide surgical targeting and therapy development for epilepsy. Included in the findings herein is the showing of a slow migrating core of high amplitude activity that can accompany seizures, and is frequently observed prior to seizures.

In some embodiments a therapeutic intervention is tested for an ability to reduce the excitatory relationship between ventral hippocampus (vHip) and mePFC. The methods disclosed herein can also be utilized to analyze the effects of agents on neurons and regions of the brain. For example, analysis of changes in excitatory relationships following exposure to one or more test compounds can performed to analyze the effect(s) of the test compounds on an individual. Such analyses can be useful for multiple purposes, for example in the development of anti-epilepsy therapies.

Parameters are quantifiable characteristics of cells, tissues and organisms, particularly components that can be accurately measured. A parameter can be, for example, the site or sites, strength, duration, speed, etc. of an electrophysiological discharge, and can be imaged by fMRI, LFP, etc. Readouts may include a single determined value, or may include mean, median value or the variance, etc. Characteristically a range of parameter readout values will be obtained for a parameter from a multiplicity of measurements. Variability is expected and a range of values for each of the set of test parameters will be obtained using standard statistical methods with a common statistical method used to provide single values.

Candidate agents of interest are for drug design are biologically active agents that encompass numerous chemical classes, primarily organic molecules, which may include organometallic molecules, inorganic molecules, genetic sequences, etc. Also of interest are therapeutic interventions, such as surgery, deep brain stimulation, optogenetics, and the like. An important aspect of the invention is to evaluate candidate therapies with preferred biological response functions.

Included are pharmacologically active drugs, genetically active molecules, etc. Compounds of interest include chemotherapeutic agents, anti-inflammatory agents, hormones or hormone antagonists, ion channel modifiers, and neuroactive agents. Exemplary of pharmaceutical agents suitable for this invention are those described in, “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Drugs Acting on the Central Nervous System; Autacoids: Drug Therapy of Inflammation; Water, Salts and Ions; etc.

Test compounds include all of the classes of molecules described above, and may further comprise samples of unknown content. Of interest are complex mixtures of naturally occurring compounds derived from natural sources such as plants. While many samples will comprise compounds in solution, solid samples that can be dissolved in a suitable solvent may also be assayed. Samples of interest include environmental samples, e.g. ground water, sea water, mining waste, etc.; biological samples, e.g. lysates prepared from crops, tissue samples, etc.; manufacturing samples, e.g. time course during preparation of pharmaceuticals; as well as libraries of compounds prepared for analysis; and the like. Samples of interest include compounds being assessed for potential therapeutic value, i.e. drug candidates.

The term samples also includes the fluids described above to which additional components have been added, for example components that affect the ionic strength, pH, total protein concentration, etc. In addition, the samples may be treated to achieve at least partial fractionation or concentration. Biological samples may be stored if care is taken to reduce degradation of the compound, e.g. under nitrogen, frozen, or a combination thereof. The volume of sample used is sufficient to allow for measurable detection, usually from about 0.1:1 to 1 ml of a biological sample is sufficient.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

As used herein, the term “genetic agent” refers to polynucleotides and analogs thereof, which-agents are tested in the screening assays of the invention by addition of the genetic agent, to a cell. The introduction of the genetic agent results in an alteration of the total genetic composition of the cell. As employed herein, the genetic agent results in the expression of a protein and is being evaluated as to its effect on one or more target pathways. The genetic agents such as DNA result in an experimentally introduced change in the genome of a cell, generally through the integration of the sequence into a chromosome. Genetic changes can also be transient, where the exogenous sequence is not integrated but is maintained as an episomal agent. RNA viruses may be employed that comprise the gene of interest and are reverse transcribed and inserted into the genome of the host cell. Genetic agents (polypeptides or polynucleotides) can also be synthesized in vitro and delivered to cells by conjugation to a moiety (e.g antennapedia 16-amino acid “Penetratin-1 peptide, available from Qbiogene) that promotes transfer of the agent into a cell of interest. The effect of a genetic agent is to increase expression of a particular gene product in the cell with the potential for the increase and/or decrease of other products in the cell.

In some instances, chemical agents of known or unknown activity are administered to an animal and the effect on seizure induction, propagation and movement assessed. These chemical agents may serve to activate a pathway, inhibit a pathway, etc., where there is interest in having a pathway other than the pathway of interest modulated and rather than using a natural factor, a chemical agent may be more convenient. The chemical agents are conveniently added in solution, or readily soluble form, and may be administered to the animal in various ways, e.g. oral, sub-cutaneous, by cannula, etc. as known in the art. Preferred chemical agent formulations consist essentially of a biologically active compound and a physiologically acceptable carrier, e.g. water, normal saline, etc.

In one embodiment a specific region of a brain of an individual is stimulated, in conjunction with combined electrophysiology, e.g. local field potentials (LFP) and functional magnetic resonance imaging (fMRI) scanning of different regions of the brain to determine functional connections between the seizure propagation zone and other regions of the brain and to image movement of a seizure. The animal may be sedated, e.g. with dexmedetomidine; and treated with a short-acting neuromuscular blocker, e.g. vecuronium, to abolish motion during imaging of seizures with simultaneous LFP-fMRI.

Suitable protocols for analysis include electrophysiology; light-induced modulation of neural activity; electroencephalography (EEG) recordings; functional imaging and behavioral analysis. Electrophysiology may include single electrode, multi electrode, and/or field potential recordings. Light-induced modulation of neural activity may include any suitable optogenetic method, as described further herein. Functional imaging may include fMRI, and any functional imaging protocols using genetically encoded indicators (e.g., calcium indicators, voltage indicators, etc.). Behavioral analysis may include any suitable behavioral assays, such as behavioral assays for arousal, memory (such as a water maze assay), conditioning (such as fear conditioning), sensory responses (responses to e.g., visual, somatosensory, auditory, gustatory, and/or olfactory cues).

The models provided herein are useful in the design and testing of therapeutic interventions, e.g. surgery, pharmacologic intervention (drug therapy), and the like, where the effect of a therapeutic intervention on seizure induction and propagation can be determined. The models are also useful in the design of drugs for epilepsy co-morbidities, e.g. the largest activity changes are in the medial prefrontal cortex (mePFC), indicating an increased excitatory relationship between ventral hippocampus (vHip) and mePFC. Therapies designed to target vHip-mePFC circuit dysfunction can reduce comorbidities associated with epilepsy, including anxiety and cognitive defects. In some embodiments these variables of seizure propagation are used to guide surgical targeting and therapy development for epilepsy. Included in the findings herein is the showing of a slow migrating core of high amplitude activity that can accompany seizures, and is frequently observed prior to seizures.

Specific findings that may be assessed include, for example, seizure onset zone (SOZ) localization with single-photon emission computed tomography (SPECT), electrophysiology and the like, where the localization detects the location of a migrating seizure core and the effect of an agent on the size, speed, duration and/or location of the migrating core. A slowly migrating core of high amplitude activity is found in the stimulated hippocampus that, in some instances, is the only detectable activity that precedes seizure generalization, pointing to a novel seizure generalization mechanism. Propagation speeds of the migrating core ranged from an average of 0.117 mm/s in non-kindled animals and 0.107 mm/s in kindled animals. Activity migrates from iVHip to iDHip, then propagates to ipsilateral cortex, contralateral hippocampus, and finally to contralateral cortex.

The induction and propagation of FBTC seizures, e.g. the effect of an agent on the size, speed, duration and/or location of the seizure. It was found that kindled seizures consistently activated bilaterally whereas non-kindled seizures preferentially activated the ipsilateral hemisphere. mThal, a region that has been implicated as a critical node for seizure generalization, was activated later than many regions of the cortex. mThal activation may be specifically analyzed.

Comparisons can be made to known antiepileptic drugs, for example gabapentin, topiramate, lamotrigine, levetiracetam, stiripentol, and rufinamide, oxcarbazepine, lacosamide, perampanel, etc.

The comparison of measurements obtained from a test agent, and a reference agent can be accomplished by the use of suitable deduction protocols, Al systems, statistical comparisons, etc. The data is compared with a database of reference results. A database of reference results can be compiled. For every reference and test pattern, typically a data matrix is generated, where each point of the data matrix corresponds to a readout from a parameter, where data for each parameter may come from replicate determinations, e.g. multiple individual seizures of the same type, etc. A data point may be quantitative, semi-quantitative, or qualitative, depending on the nature of the parameter. The readout may be a mean, average, median or the variance or other statistically or mathematically derived value associated with the measurement. The parameter readout information may be further refined by direct comparison with the corresponding reference readout. The absolute values obtained for each parameter under identical conditions will display a variability that is inherent in live biological systems and also reflects individual cellular variability as well as the variability inherent between individuals.

Classification rules are constructed from sets of training data (i.e. data matrices) obtained from multiple repeated experiments. Classification rules are selected as correctly identifying repeated reference patterns and successfully distinguishing distinct reference patterns. Classification rule-learning algorithms may include decision tree methods, statistical methods, naive Bayesian algorithms, and the like. A knowledge database will be of sufficient complexity to permit novel test agents to be effectively identified and classified. Several approaches for generating a sufficiently encompassing set of classification patterns and sufficiently powerful mathematical/statistical methods for discriminating between them can accomplish this.

### Non-Pharmacologic Therapy Design

A significant number of patients remain having seizures after treatment with antiepileptic drugs, making surgical treatments and neuromodulation therapies useful for management of epilepsy. This article reviews the current status in those two treatment modalities to drug-resistant epilepsy. The effects of surgery and surgical modifications can be tested in the models described herein to provide for greater efficacy.

Surgical modalities include resective surgery for epilepsy, particularly for mesial temporal lobe epilepsy (MTLE). However, conventional localization and resection of epileptic focus is not sufficient to achieve favorable outcomes, and can benefit from determining the site and path of migrating seizure cores. Seizure outcome after resective surgery is affected by the presence or absence of magnetic resonance imaging (MRI) lesion, pathological substrates of the associated lesion, extension and location of the epileptic focus, and selection criteria of patients for surgery.

Disconnection of lesion is a concept specific for epilepsy surgery. Complete disconnection of the epileptogenic cortices from the surrounding cortices and downstream midbrain is sufficient for control of epileptic seizures even if the pathogenic lesion is left in situ. Hemispherotomy, prefrontal disconnection, and posterior disconnection can be used. These disconnective procedures reduces surgical complications associated with extensive resection.

Intracranial recording of EEG by subdural electrodes, and depth electrodes can be used in identification of the position of a migrating seizure core for surgical purposes. Usage of frameless stereotactic navigation system allows accurate implantation of both types of electrodes simultaneously. While spikes and sharp waves are considered as epileptogenic markers in conventional EEG, ictal direct current (DC) shifts and high-frequency oscillations (HFOs) are also implicated as epileptogenic markers in wide-band EEG HFOs are usually defined as oscillatory activities higher than 80 Hz. While epileptic seizures are traditionally characterized as the hypersynchronous neuronal activity, examination of ictal firing patterns of single neurons revealed that neuronal spiking activity during seizure initiation and spread was highly heterogeneous, not hypersynchronous. And as shown herein, there is not a static core for generation of seizures.

Non-surgical intervention includes, for example, vagus nerve stimulation; deep brain stimulation (DBS) to various regions, closed loop reactive stimulation, and trigeminal nerve stimulation. Vagus nerve stimulation (VNS) therapy chronically and intermittently stimulates the left cervical vagus nerve (VN), generating afferent neural impulses that stabilize the cerebral cortex and alleviate seizures.

Intracranial neurostimulation for epilepsy includes, for example, various targets for DBS, e.g. centromedian nucleus of the thalamus, the hippocampus, the subthalamic nucleus, locus ceruleus, caudate nucleus, mammillary bodies, and the cerebellum. A closed-loop electrical stimulation system is expected to abolish seizures by giving electrical stimulation to seizure foci or other locations responding to the detected start of seizures.

### Computer Aspects

A computational system (e.g., a computer) may be used in the methods of the present disclosure to control and/or coordinate stimulus through the one or more controllers, and to analyze data from scanning of the regions of the brain. A computational unit may include any suitable components to analyze the measured images. Thus, the computational unit may include one or more of the following: a processor; a non-transient, computer-readable memory, such as a computer-readable medium; an input device, such as a keyboard, mouse, touchscreen, etc.; an output device, such as a monitor, screen, speaker, etc.; a network interface, such as a wired or wireless network interface; and the like.

The raw data from measurements, such as fMRI, LFP, and the like, can be analyzed and stored on a computer-based system. As used herein, “a computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

A variety of structural formats for the input and output means can be used to input and output the information in the computer-based systems. Such presentation provides a skilled artisan with a ranking of similarities and identifies the degree of similarity contained in the test data.

The analysis may be implemented in hardware or software, or a combination of both. In one embodiment of the invention, a machine-readable storage medium is provided, the medium comprising a data storage material encoded with machine readable data which, when using a machine programmed with instructions for using said data, is capable of displaying a any of the datasets and data comparisons of this invention. Such data may be used for a variety of purposes, such as drug discovery, analysis of interactions between cellular components, and the like. In some embodiments, the invention is implemented in computer programs executing on programmable computers, comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer may be, for example, a personal computer, microcomputer, or workstation of conventional design.

Each program can be implemented in a high level procedural or object oriented programming language to communicate with a computer system. However, the programs can be implemented in assembly or machine language, if desired. In any case, the language may be a compiled or interpreted language. Each such computer program can be stored on a storage media or device (e.g., ROM or magnetic diskette) readable by a general or special purpose programmable computer, for configuring and operating the computer when the storage media or device is read by the computer to perform the procedures described herein. The system may also be considered to be implemented as a computer-readable storage medium, configured with a computer program, where the storage medium so configured causes a computer to operate in a specific and predefined manner to perform the functions described herein. A variety of structural formats for the input and output means can be used to input and output the information in the computer-based systems of the present invention.

Further provided herein is a method of storing and/or transmitting, via computer, sequence, and other, data collected by the methods disclosed herein. Any computer or computer accessory including, but not limited to software and storage devices, can be utilized to practice the present invention. Sequence or other data (e.g., immune repertoire analysis results), can be input into a computer by a user either directly or indirectly. Additionally, any of the devices which can be used to sequence DNA or analyze DNA or analyze immune repertoire data can be linked to a computer, such that the data is transferred to a computer and/or computer-compatible storage device. Data can be stored on a computer or suitable storage device (e.g., CD). Data can also be sent from a computer to another computer or data collection point via methods well known in the art (e.g., the internet, ground mail, air mail). Thus, data collected by the methods described herein can be collected at any point or geographical location and sent to any other geographical location.

### EXPERIMENTAL

### Dissecting Seizure Networks by Imaging Individual Hippocampal Seizures Before and After Kindling

In this study, we developed and utilized a new kindling model of epileptogenesis by modifying a technique for inducing FBTC seizures that is used in epilepsy research and in anti-epileptic drug development. By repeatedly inducing electrographic seizures using cell-type specific, optogenetic stimulations, we discovered that FBTC seizures emerge and can be reliably induced thereafter for many months. We then used simultaneous electrophysiology and fMRI to investigate the impact of kindling excitatory neurons in the ventral hippocampus, the region most commonly affected in human epilepsy. We looked at the underlying hippocampal circuits altered by kindling, and conduct tests for anxiety and depression, two common epilepsy-related comorbidities, to investigate the relationship between kindling, the underlying circuit, and behavior. We directly imaged brain-wide network dynamics of single induced seizures to reveal focal and, for the first time, FBTC seizure propagation in kindled animals. In doing so, we identified key features of seizures that have significant implications for our understanding of seizure mechanisms, surgical targeting, and therapy development.

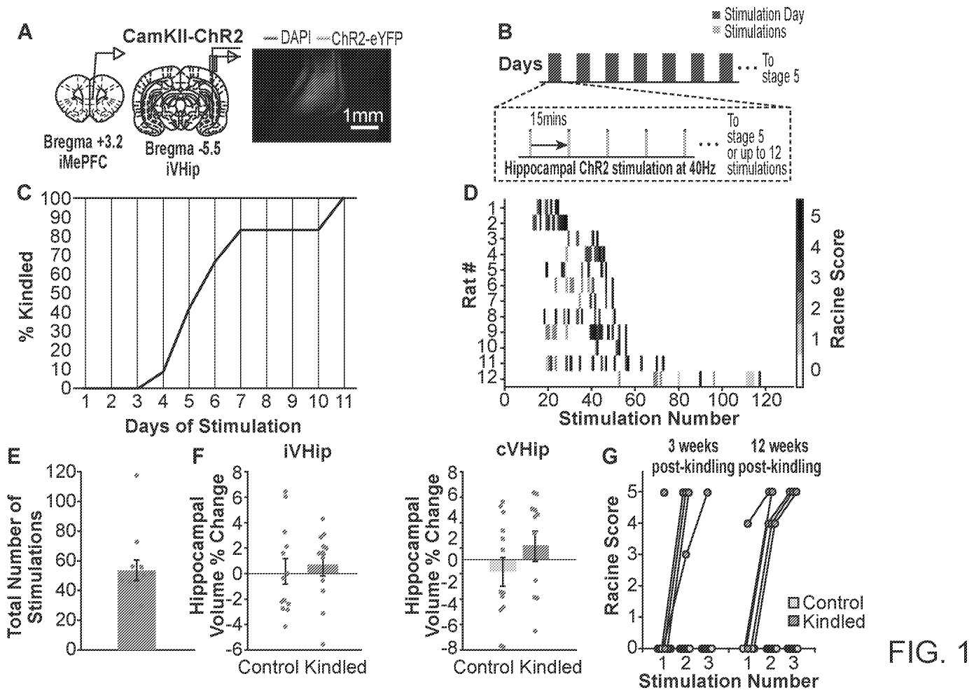

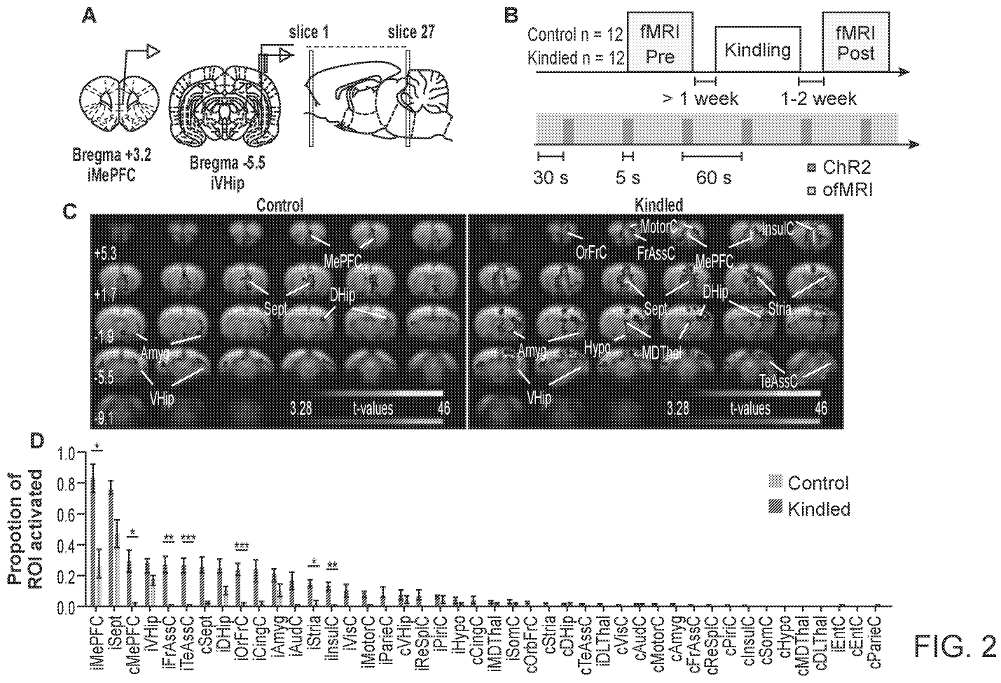

Optogenetic Kindling of Ventral Hippocampus. Kindling uses activity to remodel neuronal circuits. We developed a new kindling method and investigated the impact of repeated optogenetic stimulations of the ventral hippocampus (VHip), a region in which spontaneous seizures frequently begin in humans and in animal models. We targeted the ventral hippocampal CAMKII neurons in rats (FIG. 1A,B). Successful kindling was characterized by an evoked generalized motor seizure (Racine stage 5) within the first three stimulations of the day.

All rats reached kindling criterion by 11 days of stimulations, with 83% (10 out of 12 rats) reaching kindling criterion by day 7 (FIG. 1C). Mean number of stimulations required was 53.6 (range: 29-117, FIG. 1D), mean number of convulsive seizures (Racine stage 3-5) was 6.75 (range: 2-13), and mean number of generalized seizures (Racine stage 5) was 2.9 (range: 1-9). See FIG. 1E for a visualization of behavioral scores for each stimulation per animal.

Kindling is associated with minimal cell loss in contrast with other common epilepsy models. To assess cell loss, we measured hippocampal volumes based on anatomical MRI before and after kindling and at corresponding timepoints in non-kindled control rats. Consistent with other kindling models, we did not detect any overt hippocampal volume changes in either hemisphere (FIG. 1F). The volume change was 0.63±0.82% vs 0.14±0.96%, p=0.7, and 1.37±1.22% vs −1.03±1.27%, p=0.19 for kindled vs. non-kindled in ipsilateral and contralateral hippocampi respectively.

A key feature of kindling models is that once an animal is kindled, the effects are apparently permanent whereby motor seizures are readily evoked. To confirm that our new kindling procedure resulted in a persistent kindled state, we evaluated a subset of rats at 3 and 12 weeks after kindling and in non-kindled controls (n=5 for both groups). Motor seizures (Racine stage 5) were observed only in kindled rats and at both timepoints whereas non-kindled rats did not exhibit any overt seizure behavior (Racine stage 0) at either time-point (FIG. 1G). Taken together, we show that this optogenetic kindling procedure exhibit common key features with conventional kindling methods and provide a novel platform for our investigations into circuit remodeling and FBTC seizures.

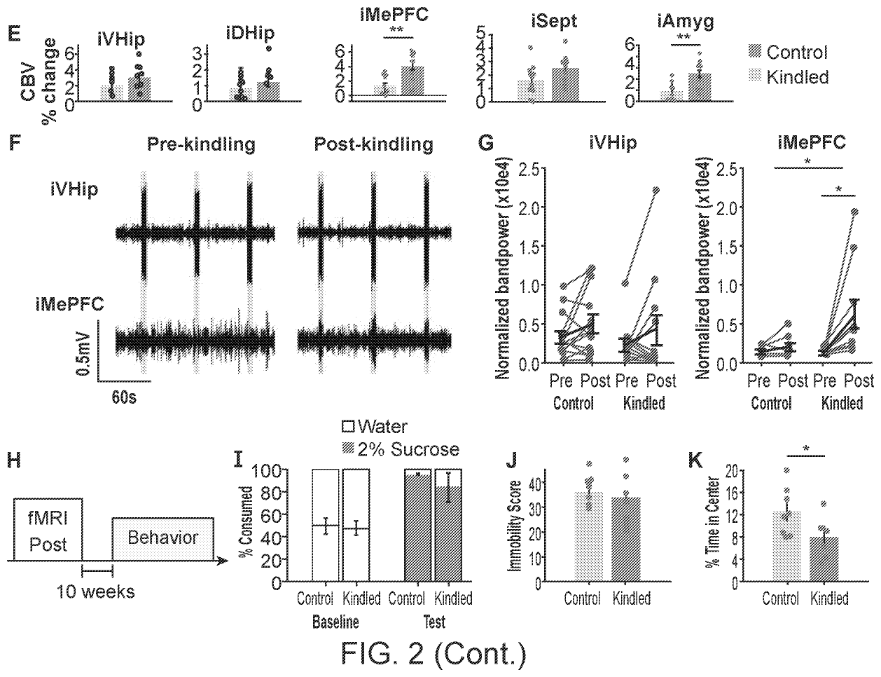

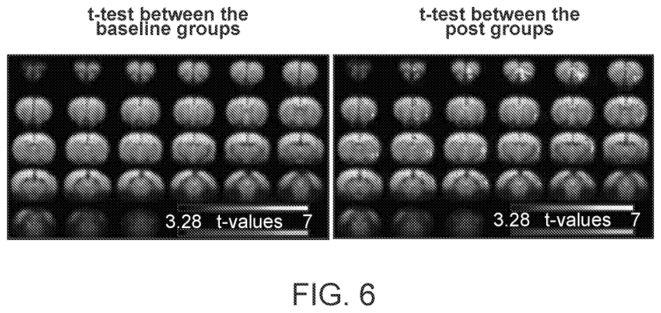

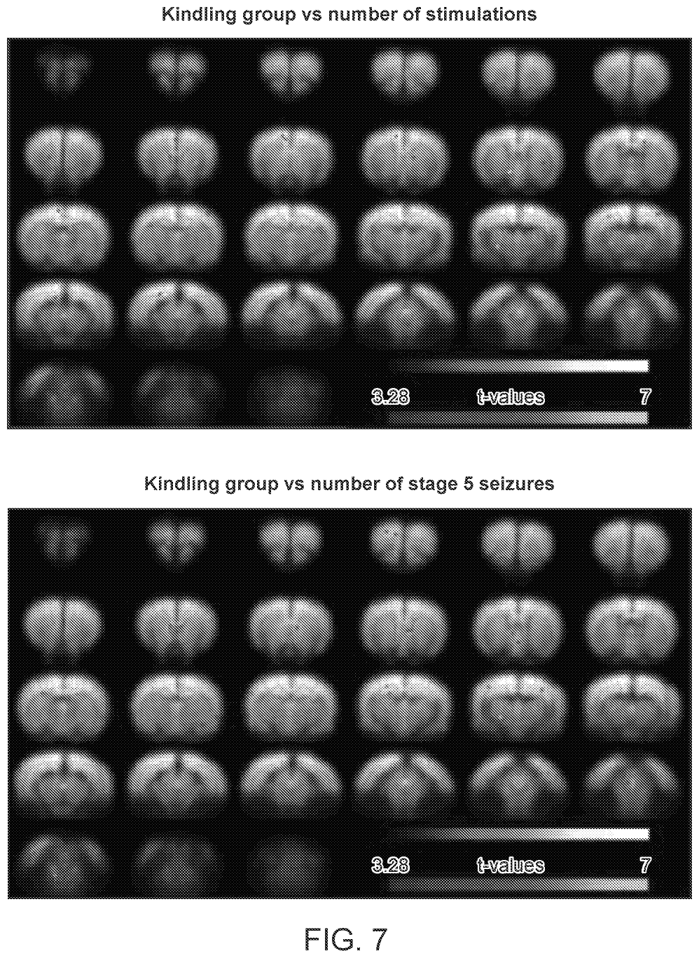

Widespread ventral hippocampal circuit remodeling and elevated anxiety after kindling. Kindling induces local structural and functional remodeling, but it remains unclear as to which downstream regions are affected. We addressed this challenge by stimulating VHip CAMKII neurons at 10 Hz and used simultaneous local field potentials (LFP) and cerebral blood volume (CBV)-fMRI to evaluate the brain-wide response with and without kindling. To visualize the brain-wide response, we generated fMRI activation maps using standard general linear model methods to identify voxels that were significantly modulated during stimulation. In non-kindled rats, stimulations resulted in activity that was localized predominantly to the following regions of the ipsilateral hemisphere: dorsal hippocampus (DHip), VHip, septum (Sept), amygdala (Amyg), and medial prefrontal cortex (MePFC) (FIG. 2C, left panel). In contrast, in kindled rats, the same stimulation resulted in activity that extended beyond these regions and include regions in the contralateral hemisphere (FIG. 2C, right panel, see FIG. 6 for group comparisons at different time points). In the kindled rats, we tested whether individual differences in the kindling procedure resulted in differences in regional activity patterns. However, inclusion of the number of stimulations for kindling or the number of stage 5 motor seizures as regressors did not yield any significant voxel-wise relationships beyond noise levels (FIG. 7).

We investigated brain-wide differences between non-kindled and kindled rats by segmenting the brain into 44 individual regions and quantifying their activation volumes (FIG. 2D). We found seven brain regions that differed between non-kindled and kindled groups following adjustment for multiple comparisons: ipsilateral MePFC (27.2±9.0% vs. 83.3±9.0%, p=0.011), contralateral MePFC (1.4±0.7% vs. 29.2±7.3%, p=0.033), ipsilateral frontal association cortex (iFrAssC) (0.24±0.24% vs. 27.1±5.6%, p=0.004), ipsilateral temporal association cortex (iTeAssC) (0.34±0.34% vs. 26.7±4.8%, p=0.0006), ipsilateral orbitofrontal cortex (iOrFrC) (1.4±0.7% vs. 23.8±3.9%, p=0.0005), ipsilateral insular cortex (ilnsC) (0.2±0.18% vs. 13.0±2.7%, p=0.0047), and ipsilateral striatum (iStria) (2.6±0.9% vs. 14.8±2.7%, p=0.011). Notably, no significant differences were observed in iVHip or iDHip (17.3±3.0% vs. 27.8±3.1%, p=0.684, 10.1±2.9% vs. 24.4±5.9%, p=0.994).

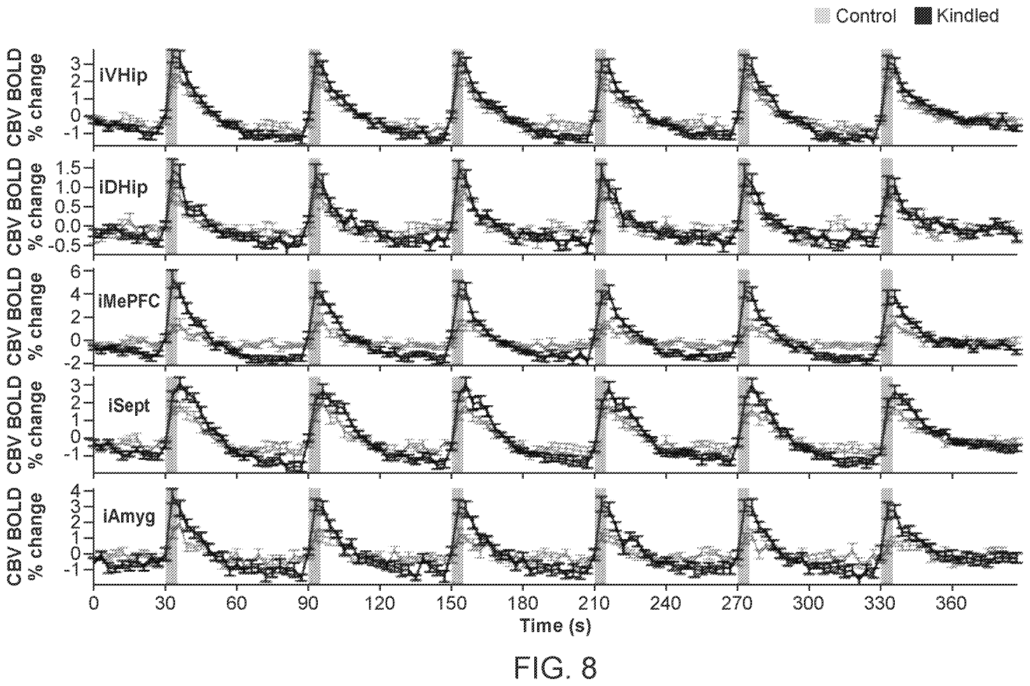

We next investigated how kindling impacted the non-kindled ventral hippocampal circuit by comparing the regional CBV-fMRI amplitudes between groups (FIG. 2E). We chose five ROIs with the largest activation volumes in the non-kindled group for comparison (FIG. 2C, left panel, see FIG. 8 for ROI time series). Following multiple comparisons correction, we did not detect differences in the amplitude of the CBV-fMRI response in iVHip (2.1±0.3% vs. 3.1±0.4%, p=0.122), iSept (1.6±0.3% vs. 2.5±0.3%, p=0.082), or iDHip (0.8±0.2% vs. 1.2±0.2%, p=0.178). However, robust differences were observed in the iMePFC (1.3±0.3% vs. 4.2±0.6%, p=0.0023), and iAmyg (1.1±0.3% vs. 3.0±0.4%, p=0.0023). These data indicate that kindling resulted in intensified responses of specific downstream regions of the non-kindled ventral hippocampal circuit.

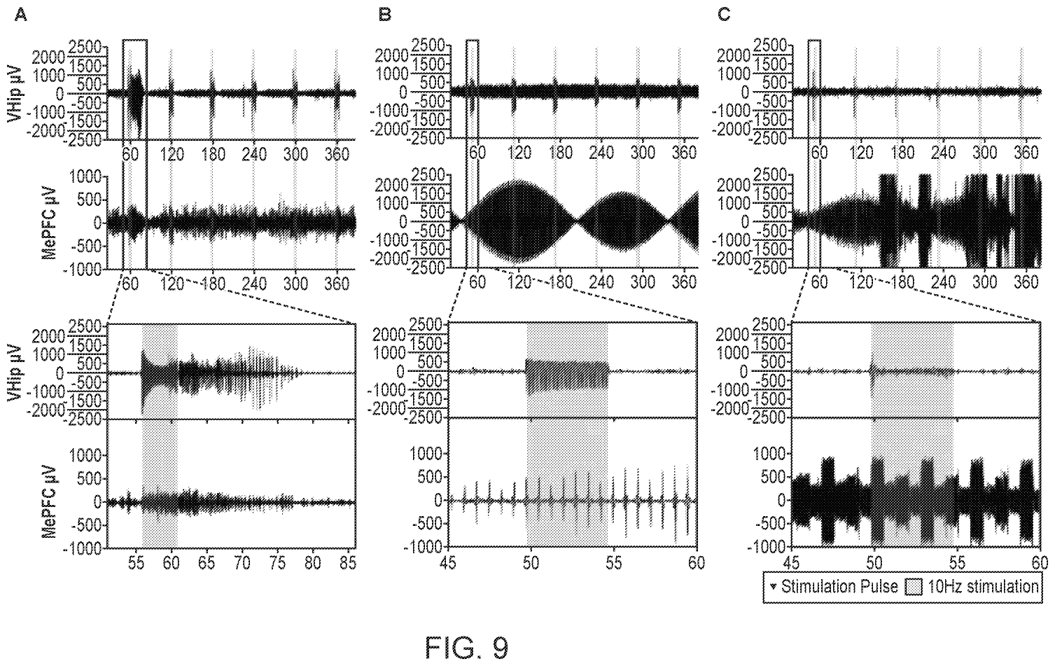

The above results indicate that optogenetic kindling in iVHip leads to more widespread (FIG. 2D) and intense (FIG. 2E) activity in iMePFC during optogenetic iVHip stimulation, measured by fMRI. We next analyzed the simultaneously acquired LFP to determine if there was a corresponding LFP signal change. Following kindling, there was a clear increase in response measured by LFP in iMePFC (FIG. 2F). We quantified this response by calculating the band power during the 5 s stimulation divided by the band power of the 5 s prior to stimulation onset. We used this metric to compare between pre and post kindling and between non-kindled and kindled groups. We did not detect any clear differences in iVHip (FIG. 2G left panel, p=0.81, n=12 per group, 2-way repeated measures ANOVA). However, in iMePFC, we found a significant interaction between group and time (FIG. 2G right panel, p=0.031, n=11 per group. Reduced number of animals for iMePFC recording was due to electrode failure, see FIG. 9). Further analysis indicated that there was a 6.4±2.6 fold increase in LFP amplitude following kindling (p=0.033, paired t-test). Taken together with the fMRI response, this suggests that kindling resulted in increased connectivity of iVHip to iMePFC.

In kindled animals, we found that there were profound changes to the iMePFC response and that there was an elevated brain response overall, but it was unclear if these changes had any long-term consequences on behavior. Because ventral hippocampus modulates emotional and affective behaviors, we investigated if ventral hippocampal kindling resulted in increased anxiety and depression: two of the most common epilepsy comorbidities with controversial mechanistic origins. Both anxiety and depression have been detected following electrical amygdala kindling. We investigated the long-term behavioral impact of our optogenetic kindling on anxiety and depression by conducting behavioral tests 12 weeks following kindling (10 weeks following the second fMRI timepoint) in a subset of animals (FIG. 2H). We use forced swim and sucrose preference tests (FIGS. 2I and J; respectively) to assess depression and the open field test to assess anxiety. Neither depression tests indicated that there were clear differences between the non-kindled and kindled animals. For forced swim test, mean immobility scores were 36.3±0.99 in non-kindled controls and 34.1±1.89 in kindled rats (FIG. 2I, p=0.303, t-test). For sucrose preference test, there was no evidence that non-kindled rats differed from the kindled rats (FIG. 2J, no significant interaction between group and habituation/sucrose test period, F=0.48 p=0.499, two-way repeated measures ANOVA, mean estimates were 96.2±0.9% vs 83.9±12.8% for non-kindled and kindled respectively). Both groups increased total volume of liquid consumed when sucrose water was introduced for the test period (non-kindled rats by 32.3±8.0 ml, p=0.005 and kindled rats by 24.7±6.9 ml, p=0.011, paired t-test). In contrast, there was evidence for an increased anxiety phenotype in kindled animals. Kindled animals spent less time in the center of the open field arena when compared to the non-kindled rats (FIG. 2K, 12.5±1.5% vs. 8.0±1.4% time in center, p=0.046, non-kindled controls vs kindled rats, t-test, n=8 and 7 respectively). Importantly, both groups of animals traveled a similar distance during the 5-minute test-period (2446±153 cm vs. 2203±197 cm, p=0.98). We find that ventral hippocampal kindling resulted in elevated anxiety but not depression, which supports the hypothesis that repeated aberrant activity can lead to underlying circuit changes resulting in anxiety.

Experiments above show that the optogenetic ventral hippocampal kindling results in increased iVHip-iMePFC connectivity and increased anxiety measured by the open field test. Notably, in a previous study, increased theta coupling between Vhip and MePFC has been observed when normal rodents were placed in an anxiogenic area (open field) compared to when they were placed in a safe area. In another study, when Arch was injected in the Vhip and an optical fiber was implanted in the MePFC, anxiety-response of normal animals placed into anxiogenic environments could be abolished by suppressing the Vhip inputs. This is interesting in the context that anxiety is one of the most common comorbidities of epilepsy. Our study establishes iVHip-iMePFC connectivity change caused by kindling initiated in hippocampus as a strong foundation for the anxiety observed in epilepsy.

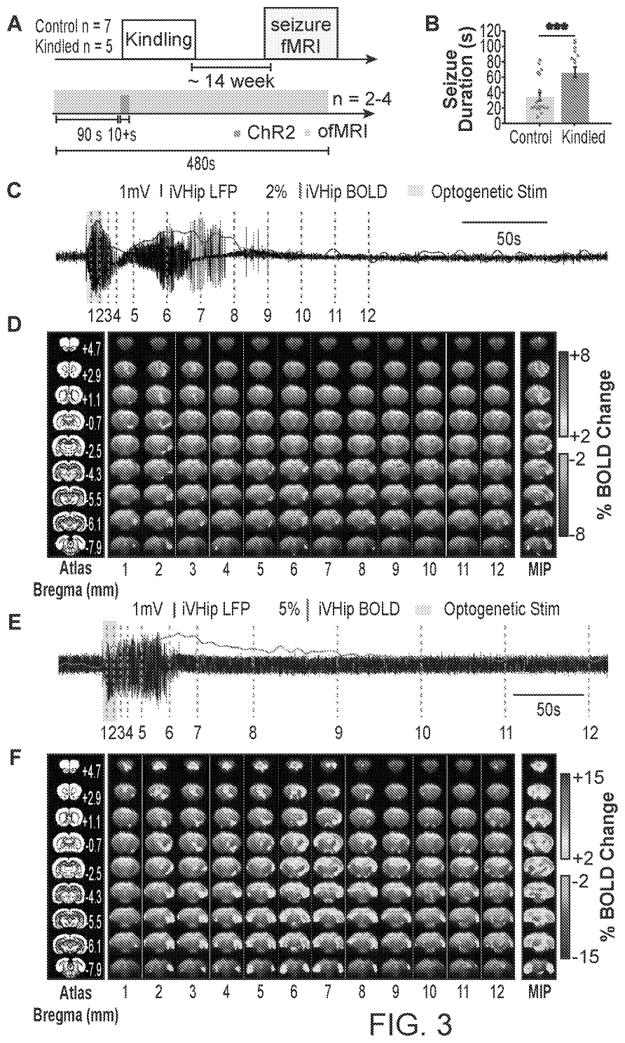

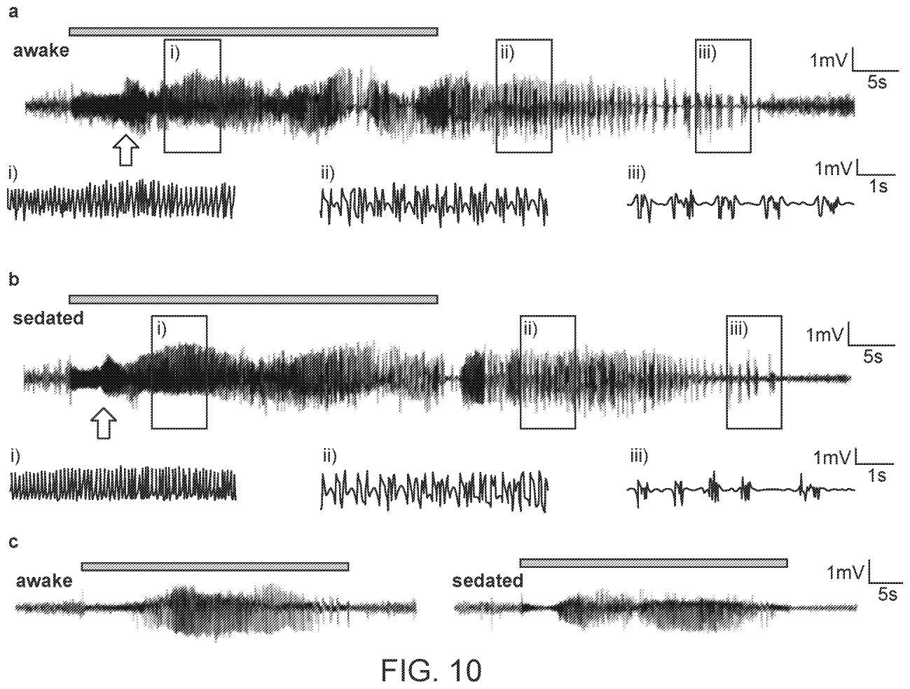

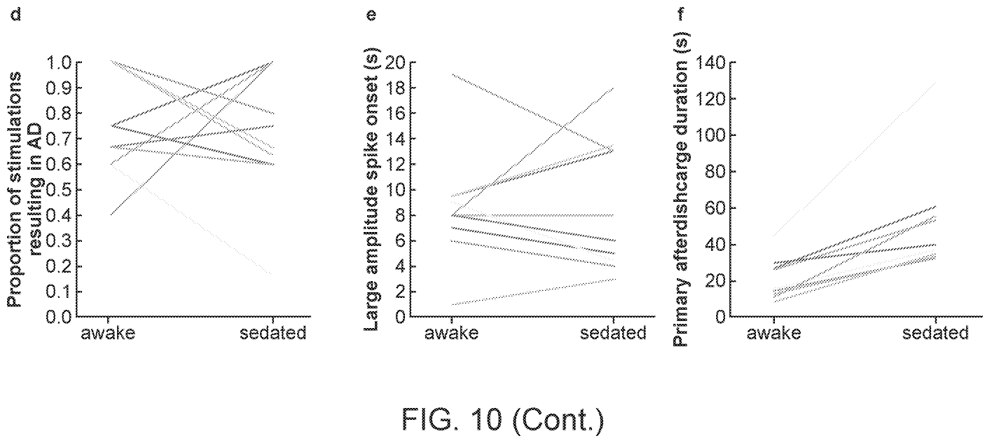

Brain-wide imaging of single seizures with simultaneous LFP-fMRI in non-kindled and kindled rats. fMRI offers brain-wide information and has been used to visualize both focal and generalized-onset seizures in humans and in animals, but its use for visualizing FBTC seizures has been challenging due to associated motor activity that result in motion artefacts. Sedation or anesthesia is typically required for animal fMRI to limit motion but their use can affect seizure activity and, in turn, the associated motor activity. We have previously demonstrated that we can reliably induce seizures under our dexmedetomidine-sedation protocol for imaging and the seizures retain similar electrophysiological characteristics in awake and sedated states (FIG. 10 for within-animal electrophysiology during non-kindled seizure in awake and sedated states). When seizures were induced in kindled animals under dexmedetomidine-sedation, stereotyped seizure behavior emerged that were reminiscent of those in awake animals indicating that motor seizure circuits were activated under sedation. Therefore, we developed a protocol based on dexmedetomidine sedation and incorporated vecuronium, a short-acting neuromuscular blocker, to abolish motion during imaging of seizures with simultaneous LFP-fMRI (FIG. 3A). Seizure duration estimated from iVHip LFP, as expected, were longer in kindled animals compared to non-kindled animals (FIG. 3B, 66.2±6.7 s vs. 35.4±5.17 s; respectively, p=0.001).

In both kindled and non-kindled animals, the iVHip BOLD signal increases corresponded to seizure-associated iVHip LFP amplitude increases, showing that BOLD successfully captured local seizure activities (FIG. 3C,E). FIG. 3 shows examples of single seizures with selected timepoints that capture activity propagation from a non-kindled and a kindled animal. A voxel-wise maximal intensity projection (MIP) over the duration of the scan provides a summary of the regions activated during a seizure (FIG. 3D,F). These MIP activity patterns bear striking similarities to those reported for focal and generalized seizures using terminal methods such as 2-deoxyglucose.

In the example of a seizure from a non-kindled animal (FIG. 3C,D), there is an initial burst of activity in the ventral hippocampal circuit, reminiscent of the 10 Hz iVHip subthreshold stimulation network (FIG. 2), followed by activity propagating to the ipsilateral dorsal hippocampus and a subsequent negative BOLD response that has been associated with post-ictal effects.

In the example of a single seizure from a kindled animal (FIG. 3E,F), there were substantially more regions with BOLD activity throughout the scan with activities now detected in cortex (FIG. 3F). Importantly, BOLD activity was seen propagating from the hippocampus to ipsilateral cortex and then contralateral cortex (FIG. 3E timepoints 5-7), clearly showing, for the first time, seizure propagation dynamics for a focal to secondary-generalized seizure across the whole brain.

For both non-kindled and kindled seizures, BOLD activity persisted beyond the end of the seizure activity detected on the iVHip LFP once activity propagates away from iVHip (FIG. 3C,D timepoints 9-10, and E,F timepoints 8-12), which clearly shows how local electrical recordings can underestimate seizure duration.

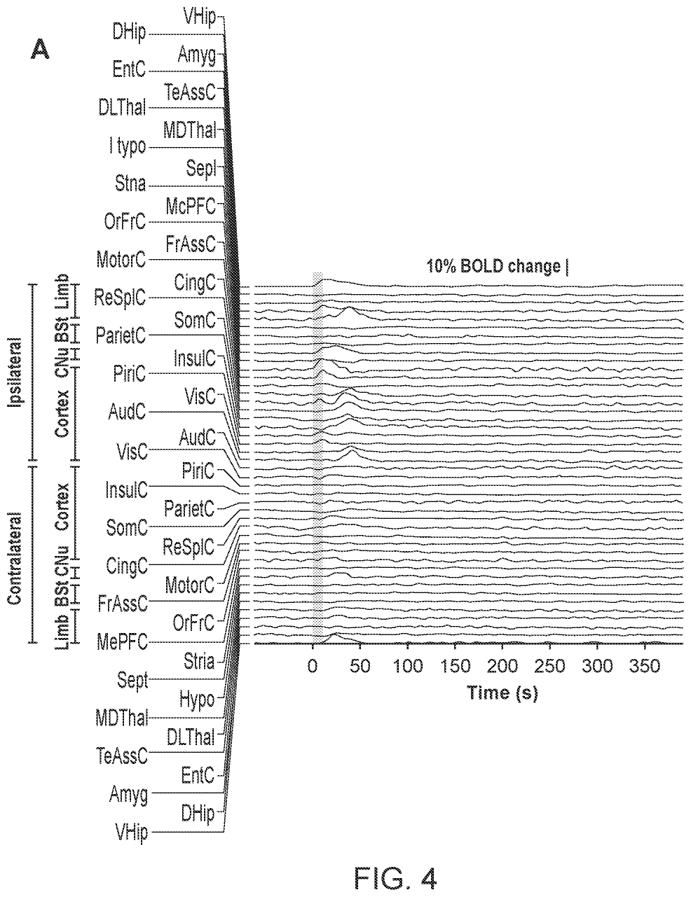

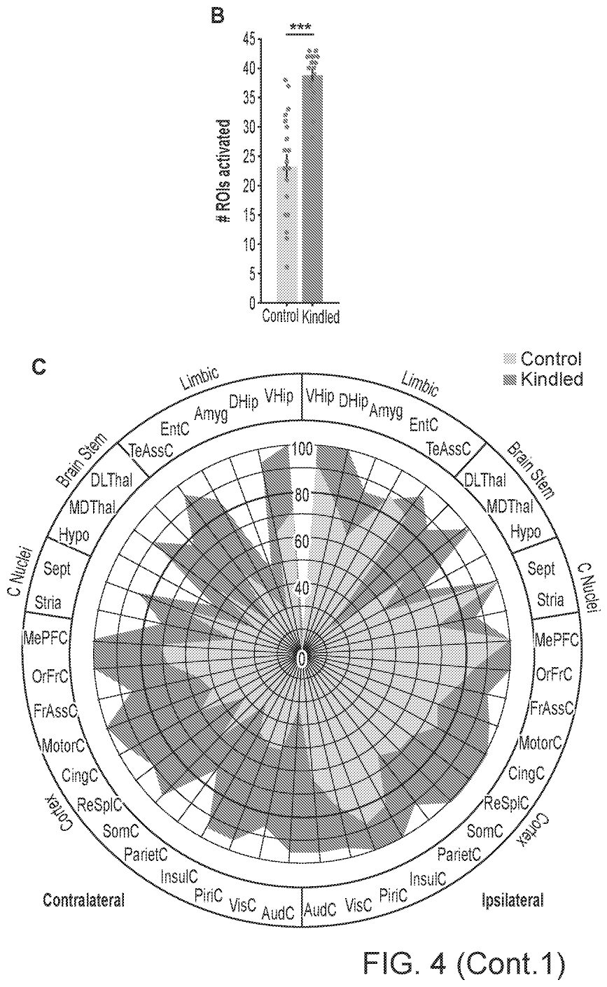

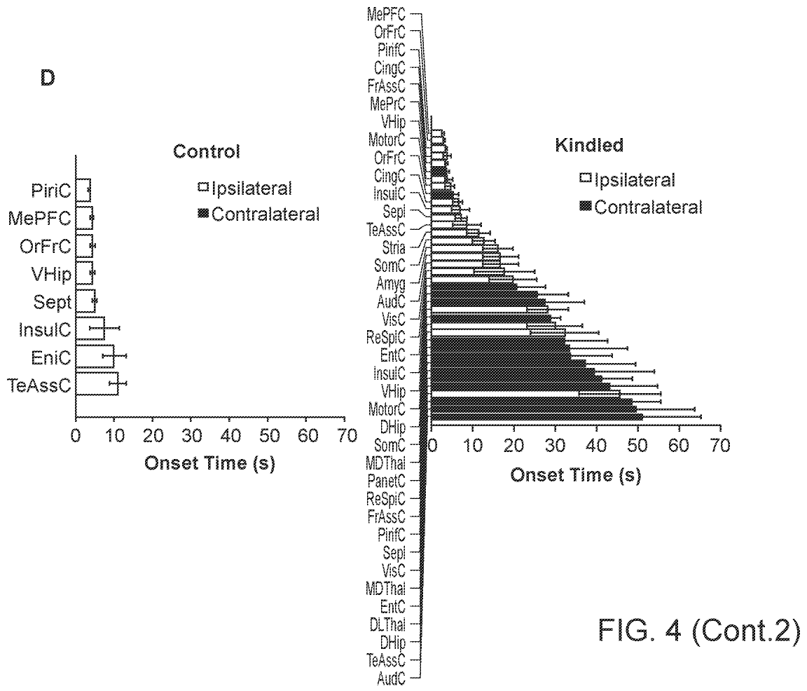

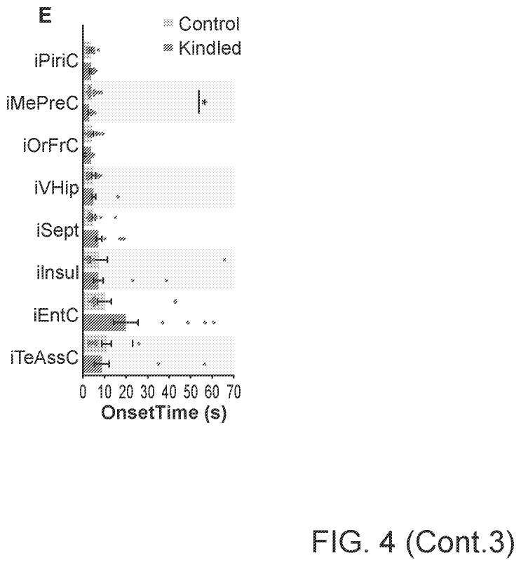

Distinct brain-wide propagation dynamics in non-kindled and kindled animals. We next compared the regional BOLD propagation patterns of non-kindled and kindled seizures by calculating regional onset times (n=20 from 7 non-kindled rats, n=17 from kindled 5 rats; respectively. One non-kindled and two kindled rats lost their head caps and could not be imaged). Using a brain atlas, mean regional responses for 44 ROIs were segmented and calculated for each seizure (FIG. 4A regional dynamics from a single seizure). Onset time was defined as the time at which BOLD activity first exceeds four standard deviations above the 60 s pre-stimulation baseline, with an additional constraint that the activity must remain above threshold for at least 5 of the subsequent 10 s after this time. We compared the number of activated ROIs and found that more regions were involved in seizures of the kindled group than in non-kindled rats (FIG. 4B, 23.4±2.0 regions vs. 38.8±1.0 regions, n=20 vs. 17, control vs. kindled, p<0.0001).

Next, we investigated the frequency of regional activation in non-kindled and kindled seizures using a modified radar plot (FIG. 4C). This visualization of the data set reveals that kindled seizures consistently activated bilaterally whereas non-kindled seizures preferentially activated the ipsilateral hemisphere.

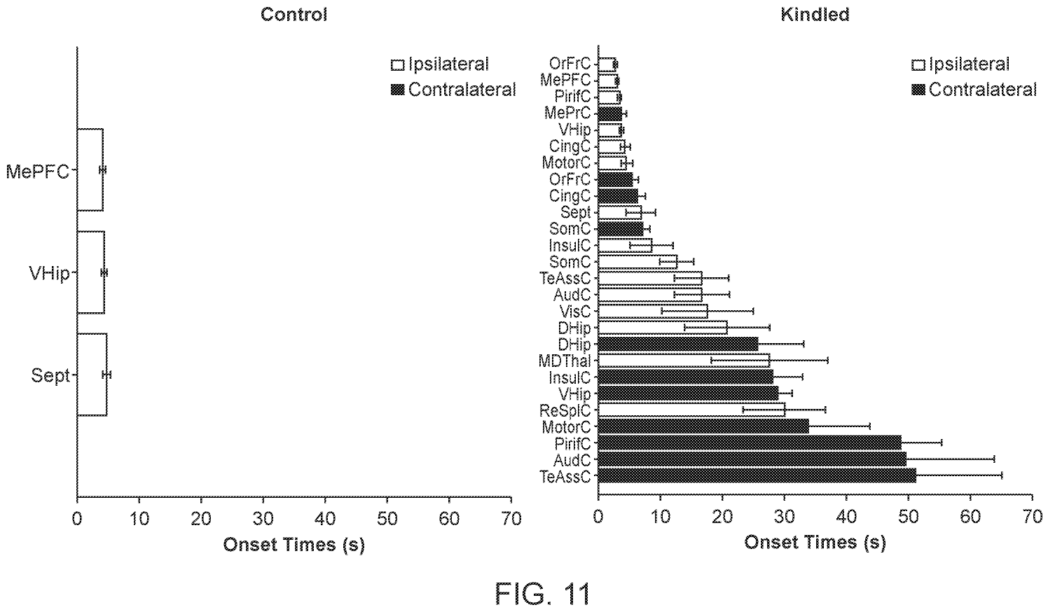

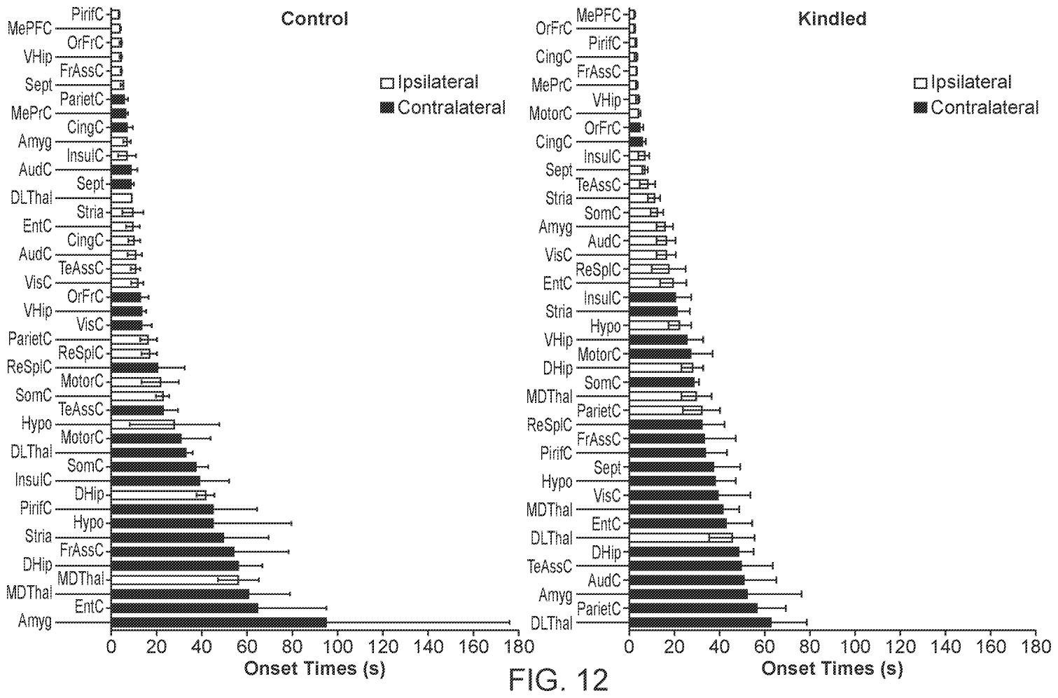

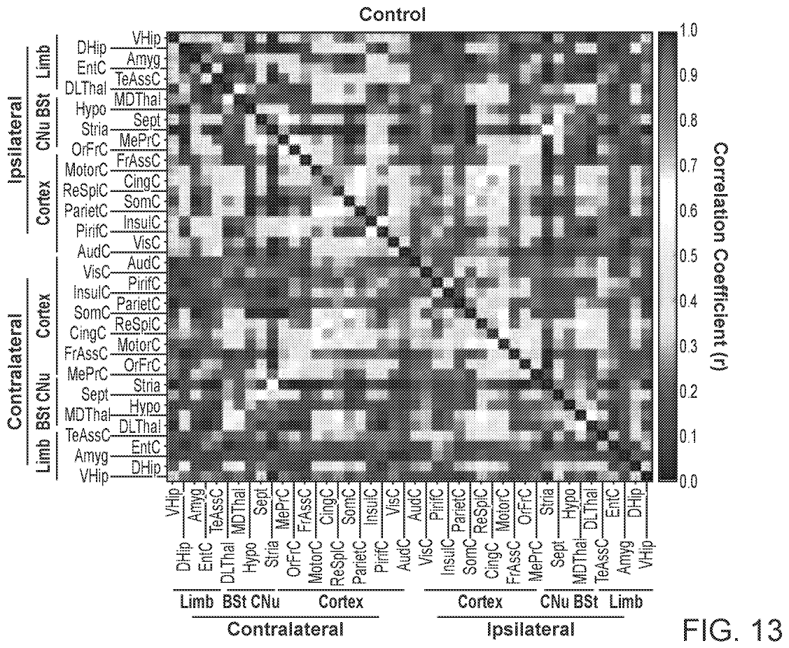

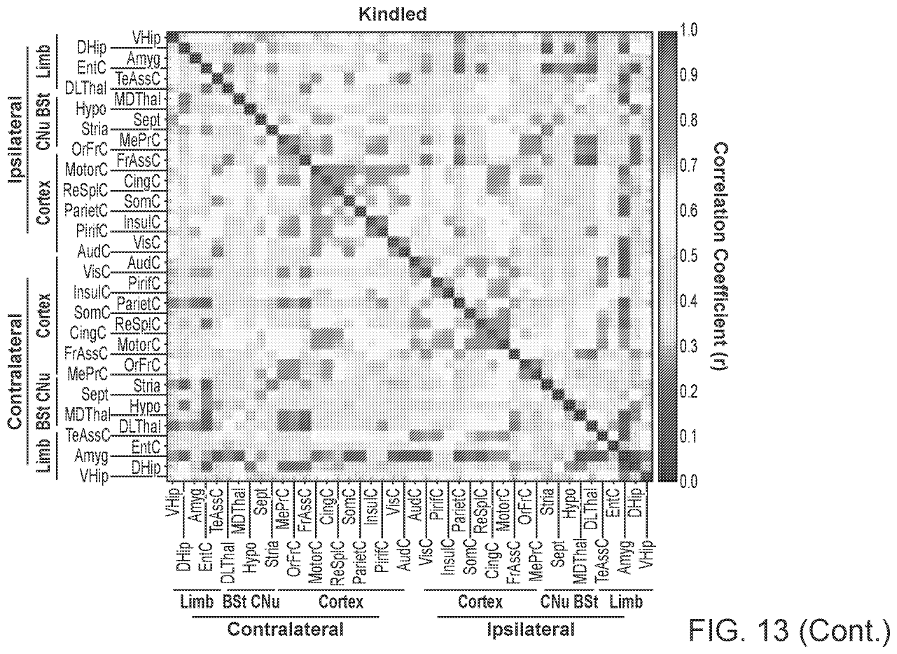

To calculate mean regional onset times for investigating activity propagation, only regions that were active in at least 80% of seizures were used (FIG. 4C, n=16-20 non-kindled and n=14-17 kindled seizures, an 80% threshold was used to obtain reliable onset time estimates. See FIG. 11, 12 for 90% and 0% thresholds, respectively). In non-kindled seizures, eight regions were active in at least 80% of the seizures, which were all from the ipsilateral hemisphere (FIG. 4D, left panel, regions were sorted from fastest to slowest): iPiriC, iMePFC, iOrFrC, iVHip, iSept, ilnsulC, ipsilateral entorhinal cortex (iEntC), iTeAssC. For kindled seizures, 38 regions from both hemispheres were active in at least 80% of the seizures (FIG. 4D, right panel). Furthermore, there was a clear pattern of propagation from the ipsilateral to the contralateral hemisphere: Of the first 20 regions with the fastest onset times, 18 regions were in the ipsilateral hemisphere. Of the remaining 18 regions, 14 were in the contralateral hemisphere (see FIG. 13 for cross correlation regional analyses for each group). Notably, mThal, a region that has been implicated as a critical node for seizure generalization, was activated later than many regions of the cortex, suggesting that mThal activation is downstream of seizure generalization.

We then compared regional onset times of the eight commonly active regions between non-kindled and kindled groups (FIG. 4E). We detected faster onset times in the iMePFC in kindled (2.8±0.26 s) compared to non-kindled seizures (4.1±0.34 s, Holm's adjusted p=0.044), while no consistent differences were observed in other regions (p>0.3 for all other regions, non-kindled vs. kindled): iVHip (4.2±0.43 s vs. 4.4±0.76 s), iOrFrC (4.2±0.51 s vs. 3.0±0.21 s), iSept (4.6±0.62 s vs. 7.1±1.34 s), ilnsC (7.1±3.94 s vs. 6.9±2.33 s), ipsilateral piriform cortex (iPiriC) (3.4±0.32 s vs. 3.4±0.33 s), iEntC (9.8±3.06 s vs. 9.7±5.7 s), and iTeAssC (10.8±2.14 s vs. 8.6±3.46 s).

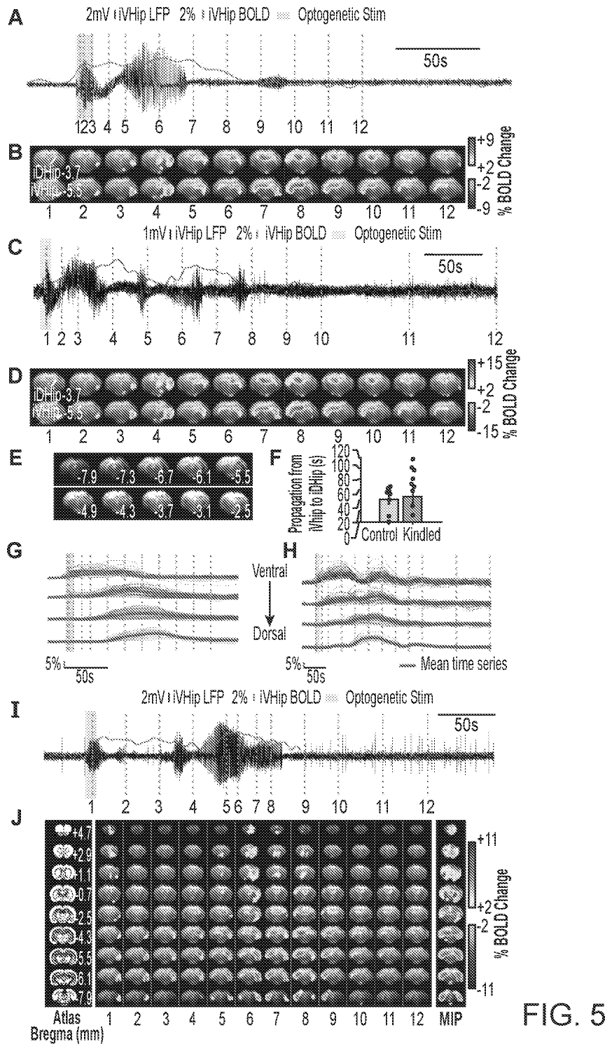

A migrating core of high amplitude activity in the hippocampus and its role in seizure generalization. During seizures, we frequently observed a slowly migrating core of high amplitude activity in the stimulated hippocampus that, in some instances, is the only detectable activity that precedes seizure generalization, pointing to a novel seizure generalization mechanism. The migrating core was observed in ipsilateral hippocampi in both non-kindled and kindled groups of animals, which suggests that the core can form with or without kindling-induced circuit reorganization, with 62% (23/37) of all seizures in 75% (9/12) of rats. A breakdown of these numbers indicate that this core was more frequently observed in kindled rats (15/17 seizures from 5/5 kindled rats) compared to non-kindled control rats (8/20 seizures in 4/7 non-kindled rats). Examples of migrating cores are shown in FIG. 5A,B in a non-kindled animal, and FIG. 5C,D in a kindled animal.

Given the migratory nature of the core, we next estimated its propagation speeds in the two groups by dividing the hippocampus into four regions from ventral to dorsal and calculated peak to peak times (FIG. 5E,G,H). Propagation speeds of the migrating core were similar in the non-kindled and kindled groups (FIG. 5F) with mean speeds of 0.117 mm/s in non-kindled rats and 0.107 mm/s in kindled rats.

While the migrating hippocampal activities in seizures of non-kindled and kindled rats shared similarities, migration to the contralateral hippocampus was only observed in kindled rats. In seizures with migrating cores, activity propagating from iDHip to cDHip was observed in 0% (0/8) in non-kindled seizures and 53% (8/15) in kindled seizures (from 4/5 rats).

Although most seizures that generalize quickly result in widespread activity (FIG. 3F), we identified a few seizures (three seizures from two kindled rats) in which the migrating hippocampal activity was the only apparent response in the brain prior to cortical activation, suggesting that the core may play a key role in seizure generalization (FIG. 5I,J. Activity migrates from iVHip to iDHip (timepoints 2-5) then propagates to ipsilateral cortex (6), contralateral hippocampus (7-8), and finally to contralateral cortex (9).

We have demonstrated brain-wide imaging of focal and of FBTC seizure dynamics and the underlying dysfunctional networks from which these seizures emerge. First, we developed a new optogenetic model of hippocampal kindling to reliably generate FBTC seizures. We then conducted brain-wide investigations of the ventral hippocampal circuit dynamics using two stimulation paradigms: a short and mild 10 Hz stimulation to evaluate underlying functional circuit changes, and a long and intense 40 Hz stimulation to evaluate seizure circuit dynamics. We found that kindling resulted in a chronic and widespread reorganization of the stimulated ventral hippocampal circuit with the largest activity changes in the mePFC. Anxiety behavior was elevated following kindled. Next, we imaged brain-wide dynamics of individual focal and FBTC seizures to reveal their distinct propagation patterns and we identified a slow migrating core of high amplitude activity that often accompany seizures. Importantly, in three kindled FBTC seizures, this core was the only activity in the brain before seizure generalization, showing that this core is a fundamental seizure propagation mechanism and has an important role in seizure generalization.

The enduring and widespread kindling-induced changes to the ventral hippocampal circuit we observed demonstrates how a specific circuit is functionally reorganized following repeated aberrant activity. It is therefore a useful animal model to study the impact of repeated epileptiform or seizure activity on the underlying circuit. Furthermore, the reorganized circuit was associated with heightened anxiety that, taken together, indicate the emergence of sensitized circuit for anxiety, one of the most common epilepsy comorbidities. We found the largest activity changes in the brain were in the mePFC, which suggests that there is an increased excitatory relationship between vHip and mePFC. Notably, these regions are two of three nodes of an anxiety circuit that shows increased theta-coupling when an animal enters an anxiolytic environment and, when theta coupling is disrupted, associated anxiety behavior was abolished. The vHip-mePFC circuit dysfunction has been observed in patients with epilepsy and in other hippocampal kindling models and the circuit dysfunction was implicated in cognitive deficits, another common epilepsy comorbidity. Taken together, these data indicate that developing therapies that target vHip-mePFC circuit dysfunction may reduce some of the most common comorbidities associated with epilepsy and enable effective treatment for conditions that can have a potentially even greater impact on quality of life than the seizures themselves.

A migrating hippocampal core as a fundamental mechanism for seizure propagation has important implications for epilepsy surgery, which requires a seizure onset zone (SOZ) to be reliably localized. Our single seizure data has a known SOZ, the ventral hippocampus, and demonstrates how a migrating core can affect SOZ localization (FIGS. 3D,F and 5J). From this data, we found that the SOZ was not active throughout the seizure, regional patterns of seizure activity can change quickly, and that regions with the highest activity are often not the SOZ. This has direct impact on standard clinical SOZ identification methods such as single-photon emission computed tomography (SPECT) and electrophysiology. SPECT uses a short-lived radioactive blood flow tracer that is manually-injected at seizure onset so any delays in injection can result in failure to detect any activity in the SOZ. Electrophysiology uses electrodes to map electrical activity during seizure and, while offering excellent temporal resolution, has positional and directional biases that can lead to misclassification of SOZ. Examples of positional bias are shown in the single seizure data in which seizure activity propagates away from the electrode implanted at the SOZ and, while seizure activity is ongoing elsewhere in the brain, seizure was no longer detected at that electrode (FIGS. 3D,F and 5J). Similarly, if an electrode is placed in the path of a migrating seizure core, seizure activity onset will reflect propagation rather than onset. Therefore, developing methods that can detect the migrating core may help to improve SOZ localization for epilepsy surgery.

The mechanisms that underlie the migrating hippocampal core are unclear and we do not know if this phenomenon occurs outside of the hippocampus. However, intrinsic hippocampal circuits were sufficient to support this core because it was observed in non-kindled as well as kindled rats. Traveling at ˜0.1 mm/s, seizure activity propagating at similar speeds have been reported in humans and in animals, and point to ionic diffusion and inhibitory restraint as potential key mechanisms. Intriguingly, recent evidence has implicated slow propagating activity as the primary mechanism for sudden unexpected death associated with epilepsy (SUDEP) and further studies are required to determine if this activity shares the same underlying mechanisms as the migrating hippocampal core.

When seizures propagated out of the hippocampus, activity followed known axonal pathways and thus implicate synaptic mechanisms. FIG. 5J is an example of propagation via synaptic transmission in which seizure activity propagates from ipsilateral to contralateral hippocampus. Synaptic seizure propagation mechanisms have been observed in a mouse model of focal cortical seizures in which activity propagated to specific regions rather than non-selectively to contiguous regions. Developing therapies that target these mechanisms can improve efficacy.

Abbreviations. ‘i’ ipsilateral, ‘c’ contralateral, ‘ChR2’ channelrhodopsin2, ‘Amyg’ amygdala, ‘AudC’ auditory cortex, ‘CingC’ cingulate cortex, ‘DHip’ dorsal hippocampus, ‘DLThal’ dorsolateral thalamus, ‘EntC’ Entorhinal cortex, ‘FrAssC’ frontal association cortex, ‘Hypo’ hypothalamus, ‘InsulC’ insular cortex, ‘MDThal’ mediodorsal thalamus, ‘mePFC’ medial prefrontal cortex, ‘MotorC’ motor cortex, ‘OrFrC’ orbitofrontal cortex, ‘ParieC’ parietal cortex, ‘PiriC’ piriform cortex, ‘RepIC’ retrosplenial cortex, ‘Sept’ septum, ‘SomC’ somatosensory cortex, ‘Stria’ striatum, ‘TeAssC’ temporal association cortex, ‘VHip’ ventral hippocampus, ‘VisC’ visual cortex.

### Material and Methods

Experimental Design. 25 adult male Sprague-Dawley rats (Charles River Laboratories) were injected with AAV-5-CAMKIIa-hChR2 (H134R)-eYFP into the right ventral hippocampus and implanted with a MRI-compatible optrode to enable simultaneous stimulation and electrophysiological recordings and the ipsilateral medial prefrontal cortex was implanted with a MRI-compatible electrode for electrophysiological recordings (REF Ben electrode paper and hippocampus paper). At least six weeks following surgery, the rats were imaged using simultaneous LFP-optogenetic fMRI (ofMRI) to investigate hippocampal circuit changes following kindling. The animals were randomly divided into two groups: an experimental kindling group and a control group (n=12, n=13 respectively). At least one week following ofMRI, the kindling group underwent kindling, and all rats were imaged again with LFP-ofMRI between one to two weeks later. A subset of rats (n=5 per group) were retested again approximately a month following LFP-of MRI to evaluate the permanence of the kindled effect. The remaining animals (n=7, n=8) were then tested for evidence of depression and anxiety using the sucrose preference test, open field test and then forced swim test ten weeks following LFP-ofMRI to minimize acute seizure effects and to evaluate the long-term consequences of kindling. Each behavior test was performed a week apart. At least a week following the end of the behavioral tests, seizures were imaged with LFP-ofMRI to investigate the nature of seizure circuits.

Surgical procedure for virus injection and implantation of optrodes and electrodes. Animal husbandry and experimental protocols were in strict accordance with the National Institutes of Health (NIH) and Stanford University's Institutional Animal Care and Use Committee (IACUC) guidelines. Animals were housed under environmentally controlled conditions, a 12-hour light-dark cycle with food and water provided ad libitum.

Briefly, rats were anesthetized using 5% isoflurane in pure oxygen and then maintained on 2-3% throughout the duration of the surgical procedure. 2 μl of AAV-5-CAMKIIa-hChR2 (H134R)-eYFP was injected into the right ventral hippocampus (AP: −5.6 mm, LR: 5.7 mm, DV: 6 mm from dura) using a using a 33-gauge needle attached to a Hamilton syringe. A syringe pump (Micro 4, World Precision Instruments, FL) was used to ensure a constant rate (150 nl/min) of administration. MRI compatible carbon fiber optrodes, constructed with 0.22 numerical aperture, 105 μm diameter step-index multimodal optical fiber (ThorLabs, Newton, N.J.) as described previously (Duffy et al., 2015), were inserted so that the tip of the electrode and fiber resided just above the injection site. Before implantation, the optrodes were checked to ensure that the percentage of light transmission was greater than 80% and light transmission to the brain was assumed to be approximately this value. A single brass screw was inserted above the cerebellum to anchor the dental cement and also to serve as ground and reference electrode. A carbon fiber electrode was implanted into the right medial prefrontal cortex (AP: +3.24 mm, LR: +1.25 mm, DV: −3.4 mm from dura at a 10° angle). Finally, the electrode wires were soldered to a DF13 connector (Hirose, Japan) and all components were secured to the skull using light-curable dental cement. Buprenorphine sustained release (1 mg/kg, s.c.) was given pre-operatively to alleviate pain and discomfort due to the procedure. Local administration of lidocaine (4%) and bupivacaine (0.25%) was also given pre- and post-operatively. To allow time for viral-induced protein expression, experiments were performed at least 6 weeks following the surgical procedure.

Optogenetic Kindling. 12 rats were kindled using the following procedure and monitored throughout using simultaneous video LFP recordings in their home cages. Animals were connected to a 473 nm (blue light) diode-pumped solid-state laser (Laserglow Technologies, Toronto, Canada) and a 16 channel BrainAmp ExG MR amplifier (Brain Products, Germany) via a fiber-optic and electrical rotary joint (Doric Lenses). Video was recorded using a Logitech C920 HD Pro webcam.

The afterdischarge threshold was first evaluated for each individual animal by gradually increasing stimulation intensity in a step-wise manner to drive electrographic seizures in the absence of behavioral seizures as assessed using the Racine scale. Starting with a 10 s train of 40 Hz stimulation with a pulse width of 7.5 ms at 1 mW, the power was increased 1 mW at a time with an interstimulus interval (ISI) of 1 min up to a maximum of 20 mW. If no afterdischarges were observed, the duration was increased by 2.5 s and power was reset to 1 mW.

After establishing the afterdischarge threshold, kindling commenced. Animals were stimulated up to a maximum of 12 stimulations a day or until the emergence of a stage 5 motor seizure, and stimulated every other day up to a maximum of 12 days of stimulations. Interstimulus interval was 15 minutes. Animals were considered to be kindled if a stage 5 motor seizure was observed within the first three stimulations of the day.

Using the same kindled criterion, the permanence of kindling was assessed in a subset of animals (five age-matched parallel controls and five kindling rats) at 3 weeks and 12 weeks following kindling by stimulating three times with an ISI of 15 minutes.

Simultaneous LFP-ofMRI Data Acquisition to assess ventral hippocampal connectivity. To assess ventral hippocampal connectivity, CAMKIIa cells in ventral hippocampus were stimulated at 10 Hz using optogenetic stimulation and assessed using simultaneous LFP-fMRI. Data acquisition was performed using the 7T horizontal-bore system (Bruker BioSpec 70/30) at the Stanford Center for Innovation in In-Vivo Imaging (SCi3). An 86 mm diameter 2-channel volume coil was used for RF excitation with a 20 mm single-loop surface coil as the RF receiver. Rats were sedated using a bolus (0.1 mg/kg, s.c.) of dexmedetomidine followed by a continuous infusion (0.05 mg/kg, i.v.) via a cannula inserted into a lateral tail vein. A single bolus of Feraheme (15 mg/kg, i.v.) was used for cerebral blood volume (CBV) weighted imaging for the enhanced contrast to noise ratio (Mandeville et al., 1998) and microvascular sensitivity (Zhao et al., 2006) that this technique offers in comparison to BOLD fMRI. fMRI acquisition was carried out approximately 15 min post contrast agent injection using a 4-shot segmented spiral readout with the following acquisition parameters: TR=0.75 ms, TE=9 ms, flip angle=30, field-of-view=32×32 mm, matrix=70×70, slice thickness=0.6 mm, number of slices=30, number of repetitions=130, number of dummy scans=4. For optogenetic stimulation, a block design was used comprising of 5 s on and 55 s off with 10 Hz, 7.5 ms pulse width for stimulation. At the end of each session, atipamazole (0.5 mg/kg, s.c.) was given to reverse the effects of dexmedetomidine. In addition to fMRI, anatomical (fast spin echo) scans were acquired for the assessment of hippocampal volumes with the following parameters: TR=4755 ms, TEeff=29.9 ms, RARE factor=8, field-of-view=30×30 mm, matrix=256×256, slice thickness=0.6 mm. For assessing hippocampal volumes, hippocampal volumes were manually drawn on the ipsilateral and contralateral hemispheres for each animal. Baseline and post-kindling measurements were made in non-kindled and kindled animals, and within animal volumes were assessed by normalizing each hippocampal volume to its baseline. An independent t-test was used to assess the differences between the two groups for each hemisphere to determine if overt hippocampal volume loss was associated with kindling.

Simultaneous LFP-ofMRI Data Acquisition to image seizure circuit dynamics. To assess brain-wide seizure circuit dynamics in non-kindled (n=7) and kindled animals (n=5), we used simultaneous LFP-fMRI with optogenetic stimulation of ventral hippocampal CAMKIIα cells. Three animals could not be imaged due to loss of their implants (non-kindled n=1, kindled n=2). Seizures were induced using a 40 Hz pulse train with a 7.5 ms pulse width at the pre-determined afterdischarge threshold as described above. Because motor seizures under the dexmedetomidine sedation protocol results in significant motion, animals were paralyzed and ventilated for seizure LFP-ofMRI. Anesthesia was induced using 5% isoflurane and maintained using 2-3% isoflurane and then rats were cannulated for drug infusion. After which the animals were cannulated and a bolus of dexmedetomidine (0.1 mg/kg, s.c.). For the remainder of the procedure, isoflurane was slowly withdrawn and a continuous infusion of dexmedetomidine (0.05 mg/kg, i.v) and vecuronium bromide (7.5 mg/kg) was used to maintain sedation and paralysis. Data acquisition was performed using the MRI system as described above. For fMRI, data were acquired using a 1-shot EPI readout with the following parameters: TR=1 s, TE=16 ms, flip angle=30, field-of-view=32×32 mm, matrix=70×70, slice thickness=0.6 mm, number of repetitions=480, number of dummy scans=4. Seizure induction began 90 s following the start of imaging. At the end of each session, atipamazole (0.5 mg/kg, s.c.) was given to reverse the effects of dexmedetomidine.

Simultaneous LFP recordings and processing. LFP recordings concurrent with the fMRI data acquisition were carried out using a 16 channel BrainAmp ExG MR amplifier (Brain Products, Germany) with a low pass filter of 1000 Hz and a sampling rate of 5000 Hz. Gradient artifacts were removed using the method implemented by Liu et al. using principal component analysis (Liu et al., 2012).

fMRI preprocessing and analysis. All fMRI data was preprocessed using SPM12, and the 10 Hz ventral hippocampal activity data was analyzed using SPM12. For preprocessing, data were first smoothed using a Gaussian kernel of 0.5 mm at full-width at half-maximum and motion corrected using a 6-parameter rigid registration. Images underwent manual brain masking and then aligned to a common space using a 12-parameter affine registration as implemented in SPM. A double gamma basis set function was used for signal convolution to the stimulation block. A general linear model (GLM) was used to generate statistical activation maps. For regional analyses, brains were automatically segmented using a common brain atlas that yielded 44 regions after which activation volumes and mean time courses were calculated for individual animals.

fMRI seizure propagation analysis. For each seizure fMRI, seizure propagation was determined at a region of interest level. The scans were registered to a brain atlas, low pass filtered at 0.1 Hz and segmented to the individual regions using the aforementioned brain atlas. A mean time course was calculated from all of the voxels within a given region. Onset time for each region was calculated by determining the time at which the signal reached four standard deviations from baseline (60 s before stimulation onset) with the additional constraint that this signal has to persist above this baseline for at least five of the subsequent 10 s.

Relationship between hippocampal connectivity and seizure induction. To investigate the relationship between hippocampal connectivity and seizures induced in that network, we calculated the conditional probability that a region was active during seizure induction given that it was active during 10 Hz non-seizure stimulation.

For the 10 Hz hippocampal connectivity data, single subject voxel level maps were generated using the aforementioned procedure and thresholded at P<0.001.

To determine the network associated with optogenetic seizure induction for each animal, we calculated the percent a voxel was activated during the first 10 s of the induction period. First, the maximum percent BOLD change was determined at the voxel level. Next, the images for each induction were first binarized using Otsu's method by applying a threshold that minimizes intraclass variance and then percent a voxel was activated across seizures within an animal was calculated to generate a map that enables evaluation of the network changes associated with optogenetic induction of seizures. For group analyses, the maps were averaged within groups to provide maps of the networks associated with optogenetic seizure induction.

With both the single subject 10 Hz hippocampal connectivity map and the single subject seizure induction network map, we proceeded to evaluate the conditional probability of a region being active during seizure induction if it was active during 10 Hz stimulation. First, all data were automatically segmented into 44 regions using the 44-region brain atlas. Next, we used a 5% activation volume threshold for both sets of data to determine if a brain region was active. After which we calculated the conditional probability at the regional level for the non-kindled and kindled groups.

Sucrose preference test. Two freshwater bottles were placed in the home cages of individually housed rats (8 controls and 7 kindling rats) for four days and their locations swapped daily to acclimate the rats to the bottles and to reduce preference for a location. The water bottles were weighed daily (9-10 am). After the acclimatization period, two freshwater bottles—one filled with water and the other filled with 5% w/v sucrose water—were placed in the cages. The water bottles were weighed and positions were swapped daily (9-10 am) for four days. The identity of the water bottles were blinded for the duration of the study. The volume of water drunk daily and the ratio between water and sucrose water was used to assess the anhedonia.

Open field test. The identity of the animals (8 controls and 7 kindling rats) were blinded for this procedure and only revealed following completion of data processing. Rats were placed individually in a custom-built chamber (1 m×1 m×0.3 m) and recorded with a Logitech C920 HD Pro webcam. Automated tracking was performed offline for the first five minutes of the recordings using Viewer3 software (Biobserve GmbH, Germany). Distance traveled and the time spent away from the edges of the arena was used to assess anxiety.

Forced swim test. The identity of the animals (8 controls and 7 kindling rats) were blinded for this procedure and only revealed following completion of data processing. Rats were placed individually in a custom-built cylinder (30 cm diameter, 90 cm high) filled up to approximately 60 cm high with water at 23-25° C. A 10-minute pre-test was performed on the day prior to the test day. Animals were recorded for the duration of the test (5 minutes) using a Logitech C920 HD Pro webcam. Animals were towel-dried and returned to their home cage. All data was analyzed offline. Animals were scored for immobility and swimming and climbing using the modified FST scoring system for which the dominant behavior at each 5 s epoch was assessed (Slattery and Cryan 2012). Immobility scores was used to assess behavioral despair.

Statistical analysis. Values are presented as mean±sem. Holm's Bonferroni method for adjustment of p-values for multiple comparisons was used where indicated. Statistical analyses were performed using SPSS 21 or custom MATLAB scripts. Statistical significance was set at P<0.05.

### Example 2

Understanding how dysfunctional brain networks give rise to seizures and impact their propagation and termination are central goals in epilepsy research and can inform the development of targeted treatments of seizures and of the comorbidities associated with epilepsy. Here, we investigate excitatory ventral hippocampal (VH) networks before and week after repeated hippocampal seizures (kindling), a procedure that results in chronic network changes, with simultaneous electrophysiology and functional MRI with optogenetic stimulation in rats. We also directly image brain-wide network dynamics of single seizures to reveal focal and, for the first time, focal to bilateral tonic-clonic seizures. We also conduct tests for anxiety and depression, two common epilepsy-related comorbidities. Finally, we report on the relationship between hippocampal networks and resulting seizure dynamics based on within-subject data. Widespread increases in excitatory VH network activity were detected following kindling. The largest change was detected in the mPFC (6.4-fold increase in LFP bandpower and 56% in activated volume following kindling). Because the VH-prefrontal cortical circuit has been implicated in anxiety and depression, we tested and found that kindled rats were more anxious (n=7.8, p=0.046). For seizure imaging, seizure duration in kindled rats was 30.7±8.4 s (p=0.001) longer than in non-kindled and 15.5±2.2 more ROIs were activated (p<0.001). Propagation network analysis indicated activity remained ipsilateral in controls while activity in kindled rats propagated gradually to cortex bilaterally. mPFC was more rapidly activated. Mediodorsal thalamus, a region implicated in seizure generalization4, was active only in kindled rats. Next, we investigated the linear relationship between the VH network and seizure networks and found that 95% of the variability was explained in controls compared to 77% in kindled rats (p<0.001, p=0.05, n=7.5). Finally, we investigated the positive predictive value of VH network activity for seizure involvement and found that in ROIs that were consistently activated it was 0.86±0.08 in controls (3 ROIs, n=7) and 0.89±0.07 in kindled rats (14 ROIs, n=5), suggesting that assessing VH activity reliably informs on seizure pathways. Taken together, these results reveal the brain-wide excitatory VH networks that underlie hippocampal seizure propagation and the long-term impact of repeated seizures on these networks.

Here, we investigate excitatory ventral hippocampal (VH) networks before and week after repeated hippocampal seizures (kindling), a procedure that results in chronic network changes, with simultaneous electrophysiology and functional MRI with optogenetic stimulation in rats. We also directly image brain-wide network dynamics of single seizures to reveal focal and, for the first time, focal to bilateral tonic-clonic seizures. We also conduct tests for anxiety and depression, two common epilepsy-related comorbidities. Finally, we report on the relationship between hippocampal networks and resulting seizure dynamics based on within-subject data.

Optogenetic Kindling. 10 of 12 (83%) rats reached kindled criterion by the seventh day of stimulation and all rats were kindled by the eleventh. The mean number of stimulations required for kindling was 53.6 (range 29-117), the mean number of convulsive seizures (Racine stage 3-5) was 6.75 (range 2-13), and mean generalized seizure number (Racine stage 5) was 2.9 (range 1-9). Successful targeting of the ventral hippocampus with ChR2-eYFP was confirmed with fluorescence microscopy.

Kindling is associated with minimal cell loss so we tested if we could detect any overt hippocampal volume loss by measuring hippocampal volumes from the anatomical MR scans acquired in the scanning sessions before and after kindling and in parallel control non-kindled rats. No significant differences in hippocampal volumes were detected in either ipsilateral of contralateral hippocampi when kindled were compared with non-kindled animals (FIG. 1F, 0.63±0.82% vs 0.14±0.96%, p=0.7, and 1.37±1.22% vs −1.03±1.27%, p=0.19, kindled vs non-kindled in ipsilateral and contralateral hippocampi respectively, mean±sem) that supports the hypothesis that kindling does not result in substantial cell loss.

To confirm the chronic effects of kindling, a subset of rats were retested at 3 and 12 weeks after kindling (n=5 for both kindled and non-kindled). All 5 kindled rats had Racine stage 5 seizures at both timepoints demonstrating the persistence of kindling, whereas non-kindled rats did not exhibit any overt seizure behavior at either time-point.

Local and brain-wide response to 10 Hz optogenetic stimulation of the ventral hippocampus with and without kindling and the impact of kindling on anxiety and depression. In non-kindled rats, 10 Hz optogenetic stimulation resulted in CBV activity that was localized predominantly to the ipsilateral hemisphere and in the dorsal and ventral hippocampus, septum, amygdala and medial prefrontal cortex. In contrast, in kindled rats, the same stimulation resulted in activity that extended beyond these regions and include regions in the contralateral hemisphere. When we compared the volume of activity of individual regions across the brain, we found consistent volume differences between the two groups of rats in seven regions following multiple comparisons correction with Bonferroni-Holm's method, ipsilateral frontal association cortex (0.24±0.24% vs 27.1±5.6%, p=0.004), temporal association cortex (0.34±0.34% vs 26.7±4.8%, p=0.0006), orbitofrontal cortex (1.4±0.7% vs 23.8±3.9%, p=0.0005), insular cortex (0.2±0.18% vs 13.0±2.7%, p=0.0047), and finally striatum (2.6±0.9% vs 14.8±2.7%, p=0.011). Neither % of ROI active in the ipsilateral ventral or dorsal hippocampus were significantly different between the groups (17.3±3.0% vs 27.8±3.1%, p=0.684, 10.1±2.9% vs 24.4±5.9%, p=0.994).

Next, we asked how the regions active in the non-kindled rats change in the kindled rats by investigating the amplitude of the fMRI response to optogenetic stimulation. We did not detect differences in the amplitude of the response in either ventral or dorsal hippocampus (non-kindled vs kindled mean±sem, p-values adjusted with Bonferroni-Holm's method for multiple comparisons), in VHip (2.1±0.3% vs 3.1±0.4%, p=0.122), in dHip (0.8±0.2% vs 1.2±0.2%, p=0.178). However, robust differences were observed in the mePFC (1.3±0.3%, 4.2±0.6%, p=0.0023), septum (1.6±0.3%, 2.5±0.3%, p=0.082), amygdala (1.1±0.3%, 3.0±0.4%, p=0.0023).

Because we measured local field potentials (LFP) in iMePFC and iVHip simultaneously with fMRI, we investigated if there was a complementary LFP response in these regions. Following kindling, there was a visually apparent increase in iMePFC. We quantified this change by calculating the bandpower response during the 5 s stimulation normalized to the bandpower in the 5 s prior to stimulation onset, and compared the responses before and after kindling with those of the non-kindled rats. We did not detect any clear differences in the ventral hippocampus (F=0.59, p=0.81, n=13 and 12 in non-kindled and kindled rats respectively, analyzed with 2-way repeated measures ANOVA). However, in the medial prefrontal cortex, we found a significant interaction between group and time (F=5.39, p=0.031, n=11 per group). Post-hoc analysis indicated that there was a 6.4±2.6 fold (mean±s.e.m.) increase following kindling (paired t-test, t=2.47, p=0.033). This taken together with the fMRI response suggests that kindling resulted in increased connectivity of the ventral hippocampus to the medial prefrontal cortex.

Because the ventral hippocampus to medial prefrontal cortical circuit has been implicated in the expression of anxiety, we investigated if the kindling procedure resulted in long-term behavioral dysfunction in a subset of animals. We tested both anxiety and depression, which are the two most common affective comorbidities associated with epilepsy. For depression, the animals underwent the forced swim and sucrose preference tests. Neither tests indicated that there were clear differences between the non-kindled and kindled animals. For forced swim test, 36.3±0.99 immobility score vs 34.1±1.89 immobility score (mean±s.e.m) in non-kindled controls vs kindled rats, p value calculated from t-test was 0.303. For sucrose preference test, we did not find evidence that that the non-kindled rats differed from the kindled rats (F=0.48 p=0.499, two-way repeated measures ANOVA, mean estimates were 96.2±0.9% vs 83.9±12.8% for non-kindled and kindled respectively). Both groups increased total volume of liquid consumed when sucrose water was introduced for the test period (non-kindled rats by 32.3±8.0 ml, p=0.005 and kindled rats by 24.7±6.9 ml, p=0.011, paired t-test). We found that kindled animals spent less time in the center of the open field arena when compared to the non-kindled rats that suggests that there is increased anxiety as a result of hippocampal kindling (12.5±1.5% vs 8.0±1.4% time in center, p=0.046. mean±s.e.m. in non-kindled controls vs kindled rats, t-test, n=8 and 7 respectively). Importantly, both groups of animals traveled a similar distance during the 5 minute test-period (2446±153 cm vs 2203±197 cm, p=0.98).

Imaging of single seizures with simultaneous LFP-fMRI in non-kindled and kindled rats.

The seizures induced in kindled animals are associated with significant motion artefacts on MRI under dexmedetomidine sedation from stereotyped behavior reminiscent of those observed in awake kindled animals. Therefore, we developed a protocol based on dexmedetomidine sedation and incorporated vecuronium, a short-acting neuromuscular blocker, to abolish motion during imaging of seizures.

In both kindled and non-kindled animals, we verified the emergence of seizures after cessation of optogenetic stimulation using LFP in the ipsilateral ventral hippocampus simultaneously-acquired with fMRI. Importantly, the ipsilateral ventral hippocampal BOLD signal followed a similar trajectory to that of the LFP, indicating that BOLD captured the seizure response. Examples of single seizures from a non-kindled and a kindled animal with LFP and single 1 s frames of normalized BOLD changes corresponding to the time points on the LFP. In the seizure from a non-kindled animal, there is an initial burst of activity in the ventral hippocampal circuit, reminiscent of the 10 Hz network, followed by propagation of activity in the ipsilateral hippocampus and a subsequent negative BOLD response. A voxel-wise maximal intensity projection (MIP) over the duration of the scan revealed the networks of high amplitude activity.

In the single seizure from the kindled animal, there was substantially more regions with BOLD activity throughout the scan with activity now detected bilaterally in cortex, indicating that seizures generalized from the initial seizure focus. Importantly, BOLD activity propagated from the initial hippocampal network to ipsilateral cortex and then contralateral cortex that indicates that, for the first time, seizure propagation dynamics for focal to secondary-generalized seizures across the whole brain. The MIP of the seizures summarizes the high amplitude activity for this experiment. Notably, for both non-kindled and kindled seizures BOLD activity persisted beyond the end of the seizure activity detected on LFP.

Seizure propagation in non-kindled and kindled animals. We next quantified BOLD activity propagation in seizures induced in non-kindled and kindled rats (n=20 from 7 rats, n=17 from 5 rats; respectively. 1 and 2 rats from the non-kindled and kindled groups lost their head caps and could not be used for this experiment). To capture propagation, we used a regional analysis using an automated segmentation atlas resulting in 44 ROIs. An example of the time series at the regional level is presented in FIG. 4A. For determining propagation, we defined regional onset as the time at which activity first reach four standard deviations above the 60s baseline prior to stimulation onset, and that the activity must remain above threshold for 5 of the subsequent 10s after this time. To verify that this procedure could distinguish between the non-kindled and kindled groups, we compared the number of detected ROIs between the two groups and confirmed that more regions were consistently activated in seizures of the kindled group than in non-kindled rats (23.4±2.0 regions vs 38.8±1.0 regions, n=20 vs 17, control vs kindled mean±sem, p<0.0001).

Next, we investigated the distribution of activity in the 44 regions in all non-kindled and kindled seizures. The arrangement of the radar plot into a mirror of the two hemispheres along the middle axis reveals that seizures in the kindled rats were consistently bilateral in nature, whereas the non-kindled rats form a subset of regions activated and those ROIs were preferentially in the ipsilateral hemisphere. For reliable estimation of onset times we used only regions that were activated in at least 80% of each seizure group (n=16-20, n=14-17 in non-kindled and kindled seizures, see supplementary figures for onsets calculated from thresholds of 90% and 0%) were included.

ROI onset times in each group were sorted from fastest to slowest and color-coded for ipsilateral and contralateral hemispheres. In non-kindled animals, eight ROIs were estimated (iPiriC, IMePFC, iOrFrC, iVHip, iSept, ilnsulC, iEntC, iTeAssC). Notably, all regions were from the ipsilateral hemisphere. For kindled seizures, activity was detected in 38 regions in which it was apparent that the ipsilateral hemisphere was activated first before activation was detected in the contralateral hemisphere. These ROIs include the eight found in the non-kindled rats. Of the first 20 regions with the fastest onset times, 18 regions were in the ipsilateral hemisphere. Of the remaining 18 regions, 14 were in the contralateral hemisphere. We next compared the onset times between the two groups in the eight regions that were consistently activated in the non-kindled brains and found that the ipsilateral mPFC was more quickly activated in the kindled than in the control (4.1±0.34 s vs 2.8±0.26 s, non-kindled control vs kindled, Holm's adjusted p=0.044) and no differences were found for the other regions (Holm's adjusted p-values>0.3): ipsilateral ventral hippocampus (4.2±0.43 s vs 4.4±0.76 s), orbitofrontal (4.2±0.51 s vs 3.0±0.21 s), septum (4.6±0.62 s vs 7.1±1.34 s), insular (7.1±3.94 s vs 6.9±2.33 s), piriform (3.4±0.32 s vs 3.4±0.33 s), entorhinal (9.8±3.06 s vs 9.7±5.7 s), and temporal association cortices (10.8±2.14 s vs 8.6±3.46 s).

The relationship between the functional networks of 10 Hz ventral hippocampal stimulation and during optogenetic seizure induction. We hypothesized that the initial circuit activation during optogenetic seizure induction recruited similar regions as those in the 10 Hz ventral hippocampal stimulations since both seizure and non-seizure inducing stimulations activated the ventral hippocampal circuitry. To evaluate the relationship between the two networks we calculated the conditional probability of an ROI being active in the seizure induction network given that it was active in the 10 Hz stimulations.

To estimate the seizure induction network for each seizure, we found voxel-wise maximum intensity value during the initial 10s seizure induction period. To determine if a voxel was active, the images were then binarized using Otsu's method, which minimizes intra-class variance (mean thresholds for non-kindled was 2.49±0.17% and for kindled groups 3.0±0.19%, mean±s.e.m, t-test p=0.32). Because we have 2-4 seizures in each animal, we calculated the frequency that each voxel was activated and then normalized to the number of seizures for group comparisons. Finally, we calculated the mean activation for each voxel at the group level to generate the seizure induction network for comparison. Visually, there were apparent and striking similarities between the seizure induction networks and those of the subthreshold stimulations at the group level. We quantified ROI activation volumes in both groups using the segmentation atlas, and sorted the volumes from largest to smallest of the kindled group. Following multiple comparisons correction, we did not detect any significant differences for any ROI but this is likely due to the small numbers of animals we had and the number of multiple comparisons involved. We investigated the number of active voxels between the two groups and found that there were more active voxels in the induction network of the kindled rats than in those of the non-kindled rats, which supports the hypothesis that following kindling the seizure induction network is larger (3562±636 voxels vs 1784±347 voxels in kindled rats and non-kindled rats respectively, t=2.64, p=0.025).

The different vascular sensitivities of the imaging procedures and the stimulation paradigms make it difficult to compare the networks at the voxel level and we therefore took an ROI approach for the calculation of conditional probabilities. For both the 10 Hz stimulation networks and the seizure induction networks, we used a 5% activation volume threshold to determine if an ROI was active or not. To check the robustness of this model, we compared the number of activated ROIS between the two experimental groups in the 10 Hz network and the optogenetic seizure induction network and found that we could detect significant differences between the groups (for 10 Hz in non-kindled=5.6±1.4 ROIs vs kindled=16.8±2.6 ROIs, t=4.10, p=0.002, for seizure induction in non-kindled=20.6±2.1 ROIs vs kindled=30.8±3.4 ROIs, t=2.69, p=0.023).

We calculated the probabilities of an ROI being active in the seizure induction network given that it was active in the corresponding 10 Hz network in individual animals to investigate the relationship between the two networks. The distribution of conditional probabilities were sorted from largest to smallest and also by their consistency of activation in 10 Hz network from most consistent to least. In the non-kindled group, iVHip and iSept were the most consistently active in the 10 Hz network and similarly in the seizure induction network. The iMePFC and iAmyg were the next set of regions that were consistently active at 10 Hz (observed in 4/7 animals) and these ROIs were also active in subsequent seizure induction. In the kindled rats, iVHip, iMePFC, iOrbFrC, iFrAssC, iSept, iStria, iTeAssC and iAmyg were active in the 10 Hz network and also active in subsequent seizure induction.

Overall, the mean conditional probability in the non-kindled animals was 0.76±0.11, and in the kindled animal was 0.89±0.05 indicating that regional activation in the 10 Hz network was similar to that of the network activated during seizure induction 12 weeks following the earlier scan. Furthermore, this supports the hypothesis that the early activity is related to seizure induction and subsequent activity arises from activation of this network.

Ipsilateral hippocampal activity propagation Following seizure induction, we observed a striking phenomenon in the ipsilateral hippocampus in a sub-population of animals in which a high amplitude cluster of activity moved from iVHip to iDHip over the course of seconds. We sought to characterize this in seizures that displayed this phenomenon (8 seizures in 4 animals from non-kindled rats, and 15 seizures from 5 kindled rats).

iVHip LFP response is overlaid with the BOLD signal from iVHip and time points are highlighted with the corresponding fMRI. For the fMRI, we highlight only two slices with iVHip and iDHip for visualization. In the non-kindled animal, activity starts in iVHip and gradually moves up the pole to iDHip. Activity persists beyond the end of the electrographic seizure. In the kindled rat, a similar pattern occurs as the activity moves up the pole. In this instance, after reaching iDHip the activity then seemingly crosses over to the contralateral DHip. Note that these seizures are from different animals to those in which the intra-hippocampal propagation is also evident.

To further characterize the intrahippocampal propagation, the ipsilateral hippocampus was segmented into four regions from the most ventral to the most dorsal. We quantified the peak to peak response in the most dorsal and most ventral regions to estimate the propagation time and found that the mean difference in non-kindled rats was 42.9 s (range 0-66 s) and in kindled rats was 46.9 s (range 0-118 s). This translates to propagation speeds of 0.07 mm/s-0.117 ms/s.

Materials and methods are as described in Example 1.

What is claimed is:

1. A model for the analysis of events associated with brain seizures, the model comprising:
a kindled animal brain that, upon stimulation, provides for neural events reflecting functional and neural circuit changes; and allows analysis of the neural events resulting from a single seizure.

2. The model of claim 1 wherein the analysis of the neural events comprises one or both of electrophysiology and magnetic resonance imaging (MRI).

3. The model of claim 1 or claim 2, wherein the analysis of the neural events is performed with simultaneous measurement of electrophysiology and functional magnetic resonance imaging (fMRI).

4. The model of any of claims 1-3, wherein the stimulation is electrical and targeted to a region of interest.

5. The model of claim 5, wherein the region of interest is the hippocampus.

6. The method of any of claims 1-5, wherein the neural events reflecting functional and neural circuit changes comprise one or more of focal to bilateral tonic-clonic (FBTC) seizures, changes in excitatory ventral hippocampal (VH) networks, changes triggered by sub-threshold stimulus, and induction of a migrating seizure core.

7. The method of claim 6, wherein the stimulation that provides for neural events reflecting functional and neural circuit changes is electrical stimulation.

8. The method of claim 7, wherein the electrical stimulation is delivered by optogenetics.

9. The method of claim 8, wherein the animal brain comprises neurons genetically engineered to comprise a light activatable polypeptide.

10. The model of claim 9, wherein the light-activatable polypeptide is a channelrhodopsin.

11. The model of claim 10, wherein the channelrhodopsin is operably linked to a promoter expressed in excitatory hippocampal neurons.

12. The model of claim 11, wherein the promoter is calmodulin-dependent kinase II alpha (CaMKIIα) promoter.

13. The method of any of claims 8-12, wherein the stimulation that provides for neural events is below the threshold to trigger seizures.

14. The model of claim 13, wherein the stimulation is from about 5 Hz to about 15 Hz.

15. The method of claim 13, wherein the stimulus is applied to evaluate underlying functional circuit changes.

16. The method of any of claims 8-12, wherein the stimulus is sufficient to trigger a seizure.

17. The model of claim 16, wherein the stimulation is from about 35 Hz to about 45 Hz.

18. The model of claim 16, wherein the stimulus is applied to evaluate seizure circuit dynamics.

19. The model of any of claims 1-18, wherein a migrating seizure core is identified.

20. The model of claim 19, wherein the migrating seizure core is used in localization of a seizure inset zone.

21. The model of claim 20, wherein the SOZ is imaged by single-photon emission computed tomography (SPECT).

22. The model of any of claims 1-21, wherein kindling is achieved using electrostimulation, optogenetics or chemical treatment.

23. The model of claim 22, wherein optogenetic kindling is performed by:
a. delivering a polynucleotide that encodes a light-activatable protein to a target neuron to a first region of the brain,
b. illuminating the first region of the brain repeatedly with a frequency and a pulse width sufficient to induce kindling; and
c. repeating step b. over the course of multiple days.

24. The model of any of claims 1-23, wherein the animal is sedated and treated with a short-acting neuromuscular blocker to abolish motion during imaging of seizures.

25. The model of claim 24, wherein the animal is sedated with dexmedetomidine and blocked with vecuronium.

26. A method for developing a therapeutic intervention for epilepsy, the method comprising:
treating the model of any of claims 1-25 with a therapeutic intervention, and determining the effect on neural events reflecting functional and neural circuit changes.

27. The method of claim 26, wherein the therapeutic intervention is surgical.

28. The method of claim 26, wherein the therapeutic intervention is electrical.

29. The method of claim 28, wherein the therapeutic intervention comprises deep brain stimulation.

30. The method of claim 28, wherein the therapeutic intervention is pharmacologic.

31. The method of any of claims 26-30, wherein the neural event comprises a seizure.

32. The method of any of claims 26-30, wherein the neural event comprises a migrating seizure core.

33. The method of any of claims 26-30, wherein the neural event comprises an epileptic comorbidity.

34. The method of any of claims 26-30, wherein the neural event comprises a focal to bilateral tonic-clonic (FBTC) seizure.

35. The method of any of claims 26-30, wherein the neural event comprises alterations in a excitatory ventral hippocampal (VH) network.
